(19) United States
(12) Patent Application Publication
(10) Pub. No.: US 2019/0192693 A1
(43) Pub. Date: Jun. 27, 2019

(54) METHODS AND VECTORS FOR TREATING CNS DISORDERS

(71) Applicants: Spark Therapeutics, Inc., Philadelphia, PA (US); The Children's Hospital of Philadelphia, Philadelphia, PA (US)

(72) Inventors: Katherine A. HIGH, Merion Station, PA (US); Beverly L. DAVIDSON, Philadelphia, PA (US)

(73) Assignees: Spark Therapeutics, Inc., Philadelphia, PA (US); The Children's Hospital of Philadelphia, Philadelphia, PA (US)

(21) Appl. No.: 16/329,697

(22) PCT Filed: Sep. 1, 2017
(86) PCT No.: PCT/US2017/049959

§ 371 Date: Feb. 28, 2019

Related U.S. Application Data
(60) Provisional application No. 62/383,274, filed on Sep. 2, 2016.

(51) Int. Cl. A61K 48/00; A61P 25/00; C12N 7/00; A61P 27/02; C07K 14/775; C12N 9/16; C12N 9/24; A61K 9/00

(52) U.S. Cl. CPC A61K 48/0075; A61P 25/00; C12N 7/00; A61P 27/02; C07K 14/775; A61K 38/00; C12Y 301/06004; C12N 9/2402; C12Y 302/01031; A61K 9/0019; A61K 48/005; C12N 9/16

(57) ABSTRACT

Methods and uses of treating a disease in a mammal are provided by administering to a mammalian non-central nervous system (CNS) cell, organ or tissue, for delivery to mammalian CNS (e.g., brain). Methods and uses of treating a disease in a mammal include, inter alia, administering to a mammalian non-ocular cell, organ or tissue for delivery to a mammalian ocular cell, organ or tissue.

### RELATED APPLICATIONS

This application claims priority to U.S. Provisional Patent Application No. 62/383,274, filed Sep. 2, 2016. The entire content of the foregoing application is incorporated herein by reference, including all text, tables and sequence listing.

### INTRODUCTION

Treatment of diseases of the central nervous system (CNS), e.g., genetic diseases of the brain such as Alzheimer's disease, remains an intractable problem. A major problem with treating brain diseases is that therapeutic proteins when delivered intravenously do not cross the blood-brain barrier, or when delivered directly to the brain, are not widely distributed. Thus, therapies for treating Alzheimer's disease need to be developed.

There are several different human apolipoprotein E (ApoE) isoforms, the presence of some of these isoforms in the brain increase the risk for Alzheimer's disease (AD), whereas the presence of other isoforms decreases the risk for AD. The presence of the ApoE ε4 isoform is a strong genetic risk factor for late-onset, sporadic AD. (Casellano et al., Sci Transl Med, 3(89):89ra57 (29 Jun. 2011).) The ApoE ε4 allele strongly increases AD risk and decreases age of onset. On the other hand, the presence of the ApoE ε2 allele appears to decrease AD risk. It is suggested that human ApoE isoforms differentially affect the clearance or synthesis of amyloid-β (Aβ) in vivo.

### SUMMARY

In certain embodiments, the invention provides a method of treating a disease in a mammal comprising administering to a mammalian non-central nervous system (CNS) cell, organ or tissue, for delivery to mammalian CNS (e.g., brain). In certain embodiments, the invention provides a method of treating a disease in a mammal comprising administering to a mammalian non-ocular cell, organ or tissue for delivery to mammalian ocular cell, organ or tissue.

The mammal can be administered a rAAV particle comprising an AAV capsid protein and a vector comprising a nucleic acid encoding a therapeutic protein inserted between a pair of AAV inverted terminal repeats in a manner effective to infect a non-CNS cell, organ or tissue. The mammal can be administered a rAAV particle comprising an AAV capsid protein and a vector comprising a nucleic acid encoding a therapeutic protein inserted between a pair of AAV inverted terminal repeats in a manner effective to infect a non-ocular cell, organ or tissue.

In certain embodiments, a non-CNS cell, organ or tissue and a non-ocular cell, organ or tissue includes a mammalian endocrine cell, organ or tissue. Exemplary endocrine cell, organ or tissue include liver cells, organ and tissue and pancreas cells, organ and tissue. In certain embodiments, a liver cell, organ or tissue is or comprises hepatocytes.

In certain embodiments, the invention provides a method of delivering a protective ApoE isoform to the CNS of a non-rodent mammal, by way of delivery or administration to a non-CNS cell, organ or tissue (e.g., not to cerebrospinal fluid (CSF) or brain) of the non-rodent mammal. In one embodiment, an rAAV particle comprising an AAV capsid protein and a vector comprising a nucleic acid encoding the protective ApoE isoform inserted between a pair of AAV inverted terminal repeats (ITRs) in a manner effective to infect non-CNS cells in the non-rodent mammal such that the non-CNS cells secrete the protective ApoE isoform into the systemic circulation (vasculature or blood vessels) of the mammal. The protective ApoE isoform in the circulation crosses the blood brain barrier and enters the CNS (e.g., cerebrospinal fluid (CSF) or brain, such as brain parenchyma).

In certain embodiments, the invention provides a method of delivering a TPP1 (tripeptidyl peptidase I), CLN3 (Battenin), PPT1 (palmitoyl protein thioesterase I), CLN6 (neuronal ceroid lipofuscinosis protein 6) or CLN8 to the CNS of a non-rodent mammal, by way of delivery or administration to a non-CNS cell, organ or tissue (e.g., not to cerebrospinal fluid (CSF) or brain) of the non-rodent mammal. In one embodiment, a rAAV particle comprising an AAV capsid protein and a vector comprising a nucleic acid encoding the TPP1, CLN3, PPT1, CLN6 or CLN8 inserted between a pair of AAV inverted terminal repeats in a manner effective to infect non-CNS cells in the non-rodent mammal such that the non-CNS cells secrete the TPP1, CLN3, PPT1, CLN6 or CLN8 into the systemic circulation (vasculature or blood vessels) of the mammal. The TPP1, CLN3, PPT1, CLN6 or CLN8 in the circulation crosses the blood brain barrier and enters the CNS (e.g., cerebrospinal fluid (CSF) or brain, such as brain parenchyma).

In certain embodiments, the invention provides a method of delivering a therapeutic protein to an ocular cell, tissue or organ of a non-rodent mammal, by way of delivery or administration to a non-ocular cell, organ or tissue of the non-rodent mammal. In one embodiment, a rAAV particle comprising an AAV capsid protein and a vector comprising a nucleic acid encoding the therapeutic protein inserted between a pair of AAV inverted terminal repeats in a manner effective to infect a non-ocular cells, tissue or organ in the non-rodent mammal such that the non-ocular cells, tissue or organ secrete the therapeutic protein into the systemic circulation (vasculature or blood vessels) of the mammal. The therapeutic protein in the circulation crosses the blood brain barrier and enters the ocular cell, tissue or organ.

In certain embodiments, the invention provides a method of transfecting a mammalian non-CNS cell, organ or tissue, for delivery to mammalian CNS (e.g., cerebrospinal fluid (CSF) or brain, such as brain parenchyma). In one aspect, a method includes delivering or administering to an endocrine cell, tissue or organ of the mammal an rAAV particle comprising an AAV capsid protein and a vector comprising a nucleic acid encoding a protective ApoE isoform inserted between a pair of AAV inverted terminal repeats in a manner effective to infect an endocrine cell, tissue or organ (e.g., liver and/or pancreas) for expression and subsequent delivery of a protective ApoE isoform to the mammalian CNS (e.g., cerebrospinal fluid (CSF) or brain, such as brain parenchyma), e.g., via the systemic circulation (vasculature or blood vessels). In another aspect, a method includes delivering or administering to the liver and/or pancreas of the mammal an rAAV particle comprising an AAV capsid protein and a vector comprising a nucleic acid encoding a protective ApoE isoform inserted between a pair of AAV inverted terminal repeats in a manner effective to infect an endocrine cell, tissue or organ (e.g., liver and/or pancreas) for expression and delivery of a protective ApoE isoform to the mammalian CNS (e.g., cerebrospinal fluid (CSF) or brain, such as brain parenchyma), e.g., via the systemic circulation (vasculature or blood vessels).

As used herein, the term “protective ApoE isoform” refers to ApoE isoforms that decrease one or more symptoms or indications of Alzheimer's disease (e.g., physical, physiological, biochemical, histological, behavioral). A protective ApoE isoform also refers to ApoE isoforms that can reduce the risk of Alzheimer's disease by at least 5%, such as 10%, 20%, 30%, 40%, 50%, 60%, 70%, 80%, 90%, 100% or more.

In certain embodiments, the invention methods provide for treating a lysosomal storage disease or disorder. In certain embodiments, the disease or disorder is a deficiency or defect in TPP1 (tripeptidyl peptidase I), CLN3 (Battenin), PPT1 (palmitoyl protein thioesterase I), CLN6 (neuronal ceroid lipofuscinosis protein 6) or CLN8 expression or activity. In certain embodiments, the disease is a neurodegenerative disease such as neuronal ceroid lipofuscinosis (NCL), such as infantile NCL, late infantile NCL, juvenile NCL (Batten disease) and adult NCL. Other lysosomal storage diseases treated in accordance with the invention affect other tissues/organs, such as Mucopolysaccharidosis (MPS IV and MPS VII), and can be treated with a rAAV particle comprising an AAV capsid protein and a vector comprising a nucleic acid encoding a therapeutic protein in accordance with the methods set forth herein.

In certain embodiments, the disease or disorder is a neurodegenerative disease such as neuronal ceroid lipofuscinosis (NCL), such as infantile NCL, late infantile NCL, juvenile NCL (Batten disease) and adult NCL inserted between a pair of AAV inverted terminal repeats in a manner effective to infect liver and/or pancreas for expression and delivery of a therapeutic protein to the mammalian CNS, e.g., via the systemic circulation (vasculature or blood vessels). In one aspect, a method includes delivering or administering to the liver and/or pancreas of the mammal an rAAV particle comprising an AAV capsid protein and a vector comprising a nucleic acid encoding a TPP1, CLN3, PPT1, CLN6 or CLN8 inserted between a pair of AAV inverted terminal repeats in a manner effective to infect liver and/or pancreas for expression and delivery of TPP1, CLN3, PPT1, CLN6 or CLN8 to the mammalian CNS (e.g., brain), e.g., via the systemic circulation (vasculature or blood vessels).

In certain embodiments, the mammal is a non-rodent mammal. In certain embodiments, the non-rodent mammal is a primate, horse, sheep, goat, pig, or dog. In certain embodiments, the mammal is human. In certain embodiments, the primate is a human. In certain embodiments, the human is a newborn, an infant, a child, a teenager or a young adult.

In certain embodiments, the mammal (e.g., human) has a CNS deficiency or disorder treatable by gene replacement or suppression therapy.

In certain embodiments, the encoded protective ApoE isoform has at least about 70% or more identity (e.g., 70-80% or 80-90%) to mammalian (e.g., primate such as a human) ApoE ε2. In certain embodiments, the encoded protective ApoE isoform has 90-100% identity to mammalian (e.g., primate such as a human) ApoE ε2.

In certain embodiments, the encoded TPP1 has at least about 70% or more identity (e.g., 70-80% or 80-90%) to a mammalian (e.g., primate such as a human) TPP1. In certain embodiments, the encoded TPP1 has 90-100% identity to mammalian (e.g., primate such as a human) TPP1.

In certain embodiments, the encoded CLN3, PPT1, CLN6 or CLN8 has at least about 70% or more identity (e.g., 70-80% or 80-90%) to a mammalian (e.g., primate such as a human) CLN3, PPT1, CLN6 or CLN8. In certain embodiments, the encoded CLN3, PPT1, CLN6 or CLN8 has 90-100% identity to mammalian (e.g., primate such as a human) CLN3, PPT1, CLN6 or CLN8.

In certain embodiments, the encoded Galactosamine-6-sulfatase has at least about 70% or more identity (e.g., 70-80% or 80-90%) to a mammalian (e.g., primate such as a human) Galactosamine-6-sulfatase. In certain embodiments, the encoded Galactosamine-6-sulfatase has 90-100% identity to mammalian (e.g., primate such as a human) Galactosamine-6-sulfatase.

In certain embodiments, the encoded beta-glucuronidase has at least about 70% or more identity (e.g., 70-80% or 80-90%) to a mammalian (e.g., primate such as a human) beta-glucuronidase. In certain embodiments, the encoded beta-glucuronidase has 90-100% identity to mammalian (e.g., primate such as a human) beta-glucuronidase.

In certain embodiments, codon-optimized nucleic acid variants encoding therapeutic proteins are employed in a vector. In certain aspects, such codon-optimized nucleic acid variants provide for increased transcription and/or translation of the encoded therapeutic protein. Such codon-optimized nucleic acid variants can exhibit increased expression, e.g., 0.5-10 fold for certain codon optimized nucleic acid variants compared non-codon optimized nucleic acid encoding therapeutic proteins.

In certain embodiments, cytosine-guanine dinucleotide (CpG) reduced nucleic acid variants encoding therapeutic proteins are employed in a vector. Such cytosine-guanine dinucleotide (CpG) reduced nucleic acid variants include variants that exhibit increased expression, e.g., 0.5-10 fold for certain CpG reduced nucleic acid variants compared non-CpG reduced nucleic acid encoding therapeutic proteins.

In one embodiment, a nucleic acid variant encoding a therapeutic protein has a reduced cytosine-guanine dinucleotide (CpG) content compared to non-CpG reduced nucleic acid encoding the protein. In particular aspects, a nucleic acid variant has at least 10 fewer cytosine-guanine dinucleotides (CpGs) than non-CpG reduced nucleic acid encoding a therapeutic protein. In additional particular aspects, a nucleic acid variant has no more than 20 CpGs; has no more than 15 CpGs; has no more than 10 CpGs; or has no more than 5 CpGs. In more particular aspects, a nucleic acid variant has at most 4 CpGs; 3 CpGs; 2 CpGs; or 1 CpG. In a further particular aspect, a nucleic acid variant encoding a therapeutic protein has no cytosine-guanine dinucleotides (CpGs).

In certain embodiments, rAAV vectors include ITRs and/or capsids based upon or having sequence identity to AAV1, AAV2, AAV3, AAV4, AAV5, AAV6, AAV7, AAV8, AAV9, AAV10, AAV11, AAV12, LK01, LK02, LK03, AAV 4-1, and/or AAV-2i8 ITRs and/or capsids. In certain embodiments, rAAV vectors include variants having less than 100% sequence identity to AAV1, AAV2, AAV3, AAV4, AAV5, AAV6, AAV7, AAV8, AAV9, AAV10, AAV11, AAV12, LK01, LK02, LK03, AAV 4-1 and/or AAV-2i8 ITRs and/or capsids. Variants include amino acid insertions, additions, substitutions and deletions. In particular aspects, variants are set forth in WO 2013/158879 (International Application PCT/US2013/037170), WO 2015/013313 (International Application PCT/US2014/047670) and US 2013/0059732 (U.S. application Ser. No. 13/594,773, discloses LK01, LK02, LK03, etc.).

In certain embodiments, rAAV vector comprises one or more ITRs and/or capsids (VP1, VP2, and/or VP3) at least 70-80%, 80-90% or 90-99% identity to AAV1, AAV2, AAV3, AAV4, AAV5, AAV6, AAV7, AAV8, AAV9, AAV10, AAV11, AAV12, LK01, LK02, LK03, AAV 4-1 and/or AAV-2i8. In certain embodiments, a rAAV vector comprises ITR(s) and/or capsid(s) (VP1, VP2 and/or VP3) having 75% or more sequence identity (e.g., 80%, 85%, 90%, 95%, 96%, 97%, 98%, 99%, 99.1%, 99.2%, 99.3%, 99.4%, 99.5%, 99.6%, 99.7%, 99.8%, etc.) to any of AAV1, AAV2, AAV3, AAV4, AAV5, AAV6, AAV7, AAV8, AAV9, AAV10, AAV11, AAV12, LK01, LK02, LK03, AAV 4-1 and/or AAV-2i8 ITRs and/or capsids. In particular aspects,

In certain embodiments, rAAV vector comprises one or more ITRs and/or capsids (VP1, VP2, and/or VP3) with 100% identity to AAV2 capsid VP1, VP2, and/or VP3 AAV1, AAV2, AAV3, AAV4, AAV5, AAV6, AAV7, AAV8, AAV9, AAV10, AAV11, AAV12, LK01, LK02, LK03, AAV 4-1 and/or AAV-2i8.

In certain embodiments, a rAAV vector comprises an AAV serotype or an AAV pseudotype comprising an AAV capsid serotype different from an ITR serotype. Pseudotype rAAV in which an AAV capsid serotype is different from an ITR serotype can be composed of ITR(s) and/or capsid(s) (VP1, VP2 and/or VP3) having 75% or more sequence identity (e.g., 80%, 85%, 90%, 95%, 96%, 97%, 98%, 99%, 99.1%, 99.2%, 99.3%, 99.4%, 99.5%, 99.6%, 99.7%, 99.8%, etc.) to any of AAV1, AAV2, AAV3, AAV4, AAV5, AAV6, AAV7, AAV8, AAV9, AAV10, AAV11, AAV12, LK01, LK02, LK03, AAV 4-1 and/or AAV-2i8 ITRs and/or capsids, provided the serotypes of the ITRs and capsids are different.

In particular aspects, an AAV vector comprises a VP1, VP2 and/or VP3 capsid sequence having 100% identity to any of AAV1, AAV2, AAV3, AAV4, AAV5, AAV6, AAV7, AAV8, AAV9, AAV10, AAV11, AAV12, LK01, LK02, LK03, AAV 4-1 and/or AAV-2i8, and one or more ITRs from a distinct serotype, e.g., an AAV2 ITR with an AAV1 capsid (AAV 2/1), an AAV6 ITR with an AAV1 capsid (AAV 6/1), an AAV2 ITR with an LK01 capsid (AAV 2/LK03), an AAV2 ITR with an AAV 4-1 capsid (AAV 2/4-1), an AAV6 ITR with an LK01 capsid (AAV 6/LK03), or an AAV6 ITR with an AAV 4-1 capsid (AAV 6/4-1).

rAAV vectors can include additional components or elements that act in cis or in trans. In particular embodiments, a vector such as rAAV vector further includes an intron, an expression control element, one or more ITRs (e.g., any of: AAV1, AAV2, AAV3, AAV4, AAV5, AAV6, AAV7, AAV8, AAV9, AAV10, AAV11, AAV12, LK01, LK02, LK03, AAV 4-1 and/or AAV-2i8 serotypes, or a combination thereof), a filler polynucleotide sequence and/or poly A signal. In particular aspects, an intron is within or flanks a nucleic acid encoding a therapeutic protein, and/or an expression control element is operably linked to the nucleic acid encoding a therapeutic protein, and/or an AAV ITR(s) flanks the 5′ or 3′ terminus of the nucleic acid encoding a therapeutic protein, and/or a filler polynucleotide sequence flanks the 5′ or 3′terminus of the nucleic acid encoding a therapeutic protein.

In particular embodiments, an expression control element comprises a constitutive or regulatable control element, or a tissue-specific expression control element or promoter. In particular aspects, an expression control element comprises an enhancer. In certain aspects, an expression control element (e.g., promoter or enhancer) confers expression in a liver cell, organ or tissue or pancreas cell, organ or tissue. In particular aspects, an expression control element (e.g., promoter or enhancer) comprises an element that confers expression in liver (e.g., a TTR promoter or mutant TTR promoter).

In certain embodiments, the invention provides a rAAV particle containing a vector comprising a nucleic acid encoding a protective ApoE isoform inserted between a pair of AAV ITRs for use in the transfection of a non-CNS cell, organ or tissue in a mammal to generate a therapeutic result in CNS. In one aspect, a use is for treating Alzheimer's disease in a mammal.

In certain embodiments, the invention provides a rAAV particle containing a vector comprising a nucleic acid encoding a TPP1 inserted between a pair of AAV ITRs for use in the transfection of a non-CNS cell, organ or tissue in a mammal to generate a therapeutic result in CNS. In one aspect, a use is for treating neuronal ceroid lipofuscinosis in a mammal.

In certain embodiments, the invention provides a rAAV particle containing a vector comprising a nucleic acid encoding a CLN3, PPT1, CLN6 or CLN8 inserted between a pair of AAV ITRs for use in the transfection of a non-CNS cell, organ or tissue in a mammal to generate a therapeutic result in CNS. In one aspect, a use is for treating Batten's disease in a mammal.

In certain embodiments, the invention provides a rAAV particle containing a vector comprising a nucleic acid encoding a Galactosamine-6-sulfatase inserted between a pair of AAV ITRs for use in the transfection of a non-ocular cell, organ or tissue in a mammal to generate a therapeutic result in ocular cell, tissue or organ. In one aspect, a use is for treating MPS IV.

In certain embodiments, the invention provides a rAAV particle containing a vector comprising a nucleic acid encoding a beta-glucuronidase inserted between a pair of AAV ITRs for use in the transfection of a non-ocular cell, organ or tissue in a mammal to generate a therapeutic result in ocular cell, tissue or organ. In one aspect, a use is for treating MPS VII.

In certain embodiments, rAAV vectors are provided, administered, delivered or used at a dose in a range from about 1×10⁸-1×10¹⁰, 1×10¹⁰-1×10¹¹, 1×10¹¹-1×10¹², 1×10¹²-1×10¹³, or 1×10¹³-1×10¹⁴ vector genomes per kilogram (vg/kg) of the mammal. In certain aspects, rAAV vectors are administered or used at a dose of less than 1×10¹² vector genomes per kilogram (vg/kg). In certain aspects, rAAV vectors are administered or used at a dose of about 5×10¹¹ vector genomes per kilogram (vg/kg) of the mammal.

In more particular aspects, amounts of rAAV vectors provided, administered, delivered or used are at least 1×10¹⁰ vector genomes (vg) per kilogram (vg/kg) of the weight of the mammal, or between about 1×10¹⁰ to 1×10¹¹ vg/kg of the weight of the mammal, or between about 1×10¹¹ to 1×10¹² vg/kg (e.g., about 1×10¹¹ to 2×10¹¹ vg/kg or about 2×10″ to 3×10¹¹ vg/kg or about 3×10¹¹ to 4×10¹¹ vg/kg or about 4×10¹¹ to 5×10¹¹ vg/kg or about 5×10¹¹ to 6×10¹¹ vg/kg or about 6×10¹¹ to 7×10¹¹ vg/kg or about 7×10¹¹ to 8×10¹¹ vg/kg or about 8×10¹¹ to 9×10¹¹ vg/kg or about 9×10¹¹ to 1×10¹² vg/kg) of the weight of the mammal, or between about 1×10¹² to 1×10¹³ vg/kg of the weight of the mammal, to achieve a desired therapeutic effect. Additional particular amounts can be in a range of about 5×10¹⁰ to 1×10¹⁰ vector genomes (vg) per kilogram (vg/kg) of the weight of the mammal, or in a range of about 1×10¹⁰ to 5×10¹¹ vg/kg of the weight of the mammal, or in a range of about 5×10¹¹ to 1×10¹² vg/kg of the weight of the mammal, or in a range of about 1×10¹² to 5×10¹³ vg/kg of the weight of the mammal, to achieve a desired therapeutic effect.

In certain embodiments, invention methods and/or uses, as set forth herein, do not induce or produce in the mammal a substantial immune response against the therapeutic protein and/or the rAAV particle. In certain aspects, the mammal does not produce a substantial humoral immune response against the therapeutic protein and/or the rAAV particle. In certain aspects, a substantial immune response (e.g., humoral) against the therapeutic protein and/or the rAAV particle is not produced for at least 1, 2, 3, 4, 5, 6, 7, 8, 9, 10, 11, 12, 13 or 14 continuous days, weeks or months.

A substantial immune response in the context of treatment is considered to be that which significantly reduces efficacy. Thus, in the case of a CNS disorder or disease, it would be a substantial immune response if there is no detectable efficacy in treating CNS disorder or disease. Accordingly, a substantial immune response does not mean an immune response that is minimal or does not result in a loss or significant reduction in efficacy in the context of treatment.

In certain embodiments, the mammal does not develop a detectable immune response against the therapeutic protein and/or the rAAV particle. In certain embodiments, the mammal does not develop a detectable humoral immune response against therapeutic protein and/or the rAAV particle.

In certain embodiments, the mammal does not develop an immune response (e.g., humoral) against therapeutic protein and/or the rAAV particle sufficient to block a therapeutic effect of the therapeutic protein. In certain aspects, the mammal does not produce an immune response (e.g., humoral) against the therapeutic protein and/or the rAAV particle sufficient to block the therapeutic effect for at least 1, 2, 3, 4, 5, 6, 7, 8, 9, 10, 11, 12, 13 or 14 continuous days, weeks or months.

In certain embodiments, empty capsids can be included in rAAV vectors, methods and uses. If desired, AAV empty capsids can be added to rAAV vector preparations, or administered separately to a subject in accordance with the methods and uses herein.

In certain embodiments, AAV empty capsids are formulated with rAAV vectors and/or administered to a mammal. In particular aspects, AAV empty capsids are formulated with less than or an equal amount of vector (e.g., about 1.0 to 100-fold rAAV vectors to AAV empty capsids, or about a 1:1 ratio of rAAV vectors to AAV empty capsids). In other particular aspects, rAAV vectors are formulated with an excess of AAV empty capsids (e.g., greater than 1 fold AAV empty capsids to rAAV vectors, e.g., 1.0 to 100-fold AAV empty capsids to rAAV vectors). Optionally, a mammal with low to negative titer AAV NAb can receive lower amounts of empty capsids (1 to 10 fold AAV empty capsids to rAAV vectors, 2-6 fold AAV empty capsids to rAAV vectors, or about 4-5 fold AAV empty capsids to rAAV vectors).

In certain embodiments, rAAV vectors, methods and uses include an excess of empty capsids greater than the dose or amount of rAAV vectors (i.e., those containing a nucleic acid encoding a therapeutic protein) in the composition. A ratio of empty capsids to rAAV vectors can be about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10 to 1, or some other ratio.

In some embodiments, empty capsids comprise the same VP1, VP2, and VP3 capsid proteins that are present in the rAAV vectors. In other embodiments, empty capsids comprise VP1, VP2 and VP3 proteins having a different amino acid sequence than those found in the rAAV vectors. Optionally, if the capsid proteins of the empty capsids and capsids of the rAAV vectors are not identical in sequence, they will be of the same serotype.

In certain embodiments, empty capsids are included in rAAV vectors, methods and uses, in which rAAV vectors are in particular doses or amounts. In some embodiments, a dose of rAAV vectors is about 1×10¹⁰ to 1×10¹¹ vg/kg of the weight of the mammal, or between about 1×10¹¹ to 1×10¹² vg/kg (e.g., about 1×10¹¹ to 2×10¹¹ vg/kg or about 2×10¹¹ to 3×10¹¹ vg/kg or about 3×10¹¹ to 4×10¹¹ vg/kg or about 4×10¹¹ to 5×10¹¹ vg/kg or about 5×10¹¹ to 6×10¹¹ vg/kg or about 6×10¹¹ to 7×10¹¹ vg/kg or about 7×10¹¹ to 8×10¹¹ vg/kg or about 8×10¹¹ to 9×10¹¹ vg/kg or about 9×10¹¹ to 1×10¹² vg/kg) of the weight of the mammal, or between about 1×10¹² to 1×10¹³ vg/kg of the weight of the mammal, and empty capsids.

In some embodiments, a dose or amount of rAAV vector, or a method or use employing a dose or amount of rAAV vector optionally has an excess of empty capsids. In some embodiments, a dose of rAAV vectors is about 1×10¹⁰ to 1×10¹¹ vg/kg of the weight of the mammal, or between about 1×10¹¹ to 1×10¹² vg/kg (e.g., about 1×10¹¹ to 2×10¹¹ vg/kg or about 2×10¹¹ to 3×10¹¹ vg/kg or about 3×10¹¹ to 4×10¹¹ vg/kg or about 4×10¹¹ to 5×10¹¹ vg/kg or about 5×10¹¹ to 6×10¹¹ vg/kg or about 6×10¹¹ to 7×10¹¹ vg/kg or about 7×10¹¹ to 8×10¹¹ vg/kg or about 8×10¹¹ to 9×10¹¹ vg/kg or about 9×10¹¹ to 1×10¹² vg/kg) of the weight of the mammal, or between about 1×10¹² to 1×10¹³ vg/kg of the weight of the mammal, and an excess of empty capsids. The excess of capsids over each dose or amount of rAAV vector can be about 1.5 to 100-fold AAV empty capsids to rAAV vectors. If desired, the ratio of empty capsids to rAAV vectors can be about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10 to 1.

In certain embodiments, the administering or delivery is by way of infusion or injection into the systemic circulation of the subject. In certain embodiments, the administering or delivery is by way of intravenous or intra-arterial infusion or injection into the systemic circulation of the subject. In certain embodiments, the administering or delivery is by way of infusion or injection into the hepatic portal vein of the subject. In certain embodiments, the administering or delivery is by way of an implant or a pump that provides infusion or injection into the systemic circulation of the subject, or that provides intravenous or intra-arterial infusion or injection into the systemic circulation of the subject, or that provides infusion or injection into the hepatic portal vein of the subject.

### DETAILED DESCRIPTION

The invention is based at least in part on development of a rAAV vector that when administered to a non-central nervous system (CNS) or non-ocular cell, tissue or organ is able to infect a non-central nervous system (CNS) or non-ocular target cell, tissue or organ for expression and provide subsequent delivery of the protein encoded by the heterologous nucleic acid to the mammalian CNS or an ocular cell, tissue or organ. Expression of the encoded protein by the non-central nervous system (CNS) or non-ocular cell, tissue or organ leads to secretion of the encoded protein into the systemic circulation which in turn delivers the encoded protein to the mammalian CNS and/or the ocular cell, tissue or organ. Accordingly, the invention provides methods of delivering proteins to the mammalian CNS and/or the ocular cell, tissue or organ without direct administration to the mammalian CNS and/or the ocular cell, tissue or organ, i.e., to a cell, tissue or organ other than the mammalian CNS or the ocular cell, tissue or organ. Such target cells, tissues and organs include endocrine cells, tissues and organs. Particular non-limiting examples include the liver (e.g., hepatocytes).

Adeno associated virus (AAV) is a small nonpathogenic virus of the parvoviridae family. AAV is distinct from the other members of this family by its dependence upon a helper virus for replication. In the absence of a helper virus, AAV may integrate in a locus specific manner into the q arm of chromosome 19. The approximately 5 kb genome of AAV consists of one segment of single stranded DNA of either plus or minus polarity. The ends of the genome are short inverted terminal repeats which can fold into hairpin structures and serve as the origin of viral DNA replication. Physically, the parvovirus virion is non-enveloped and its icosohedral capsid is approximately 20-30 nm in diameter.

As will be appreciated by one of ordinary skill in the art, an AAV virion consists of three related proteins referred to as VP1 protein and two shorter proteins, called VP2 and VP3 that are essentially amino-terminal truncations of VP1. Depending on the capsid and other factors known to those of ordinary skill, the three capsid proteins VP1, VP2 and VP3 are typically present in a capsid at a ratio approximating 1:1:10, respectively, although this ratio, particularly of VP3, can vary significantly and should not to be considered limiting in any respect.

The ends of the AAV genome have short inverted terminal repeats (ITR) which have the potential to fold into T-shaped hairpin structures that serve as the origin of viral DNA replication. Within the ITR region two elements have been described which are central to the function of the ITR, a GAGC repeat motif and the terminal resolution site (trs). The repeat motif has been shown to bind Rep when the ITR is in either a linear or hairpin conformation. This binding serves to position Rep68/78 for cleavage at the trs which occurs in a site- and strand-specific manner. In addition to their role in replication, these two elements appear to be central to viral integration. Contained within the chromosome 19 integration locus is a Rep binding site with an adjacent trs. These elements have been shown to be functional for locus specific integration.

AAV are useful as gene therapy vectors as they can penetrate cells and introduce nucleic acid/genetic material so that the nucleic acid/genetic material may be stably maintained in cells. In addition, these viruses can introduce nucleic acid/genetic material into specific sites, for example, such as a specific site on chromosome 19. Because AAV are not associated with pathogenic disease in humans, AAV vectors are able to deliver heterologous polynucleotide sequences (e.g., therapeutic proteins and agents) to human patients without causing substantial AAV pathogenesis or disease.

Accordingly, rAAV vectors, including serotypes and variants provide a means for delivery of nucleic acid sequences into cells ex vivo, in vitro and in vivo, which can encode proteins such that the cells express the encoded proteins. For example, a recombinant AAV vector can include a heterologous nucleic acid encoding a desired protein or peptide (e.g., a protective apoE isoform). Vector delivery or administration to a subject (e.g., mammal) therefore provides the encoded protein to the subject.

As used herein, the term “recombinant,” as a modifier of AAV vectors, as well as a modifier of sequences such as recombinant nucleic acids and polypeptides, means that the compositions (e.g., AAV or sequences) have been manipulated (i.e., engineered) in a fashion that generally does not occur in nature. A particular example of a recombinant AAV vector would be where a nucleic acid that is not normally present in the wild-type viral (e.g., AAV) genome (“heterologous”) is inserted within the viral genome. A “recombinant” AAV vector is distinguished from an AAV genome, since all or a part of the viral genome has been replaced with a non-native sequence with respect to the AAV genomic nucleic acid such as a heterologous nucleic acid sequence. Although the term “recombinant” is not always used herein in reference to AAV vectors, as well as sequences such as nucleic acids and polypeptides, recombinant forms of AAV, and sequences including nucleic acids and polypeptides, are expressly included in spite of any such omission.

Typically for AAV one or both inverted terminal repeat (ITR) sequences of AAV genome are retained in the AAV vector. Incorporation of a non-native sequence (e.g., protective apoE isoform) therefore defines the AAV vector as a “recombinant” AAV (rAAV) vector.

A recombinant AAV vector can be packaged—referred to herein as a “particle” for subsequent infection (transduction) of a cell, ex vivo, in vitro or in vivo. Where a recombinant AAV vector sequence is encapsidated or packaged into an AAV particle, the particle can also be referred to herein as a “rAAV.” Such particles include proteins that encapsidate or package the vector genomes, and in the case of AAV, capsid proteins.

An “AAV viral particle” or “AAV particle” refers to a viral particle composed of at least one AAV capsid protein (typically all of the capsid proteins of an AAV) and an encapsidated nucleic acid, referred to as a vector genome. If the particle comprises heterologous nucleic acid, it is typically referred to as “rAAV.”

An AAV vector “genome” refers to the portion of the recombinant plasmid sequence that is ultimately packaged or encapsidated to form an AAV particle. In cases where recombinant plasmids are used to construct or manufacture recombinant AAV vectors, the AAV vector genome does not include the portion of the “plasmid” that does not correspond to the vector genome sequence of the recombinant plasmid. This non vector genome portion of the recombinant plasmid is referred to as the “plasmid backbone,” which is important for cloning and amplification of the plasmid, a process that is needed for propagation and recombinant AAV production, but is not itself packaged or encapsidated into rAAV particles.

In particular embodiments, rAAV vectors include capsids derived from AAV1, AAV2, AAV3, AAV4, AAV5, AAV6, AAV7, AAV8, AAV9, AAV10, AAV11, Rh10, Rh74 and AAV-2i8, as well as variants (e.g., capsid variants, such as amino acid insertions, additions and substitutions) thereof. rAAV vector serotypes and variants include capsid variants (e.g., LK03, 4-1, etc.).

rAAV serotypes and rAAV variants (e.g., capsid variants such as LK03, 4-1) may or may not be distinct from other AAV serotypes (e.g., distinct from VP1, VP2, and/or VP3 sequences). As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV variants including capsid variants may not be serologically distinct from a reference AAV or other AAV serotype, they differ by at least one nucleotide or amino acid residue compared to the reference or other AAV serotype.

Under the traditional definition, a serotype means that the virus of interest has been tested against serum specific for all existing and characterized serotypes for neutralizing activity and no antibodies have been found that neutralize the virus of interest. As more naturally occurring virus isolates of are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

Recombinant AAV vector (e.g., rAAV), as well as methods and uses thereof, include any viral strain or serotype. As a non-limiting example, a recombinant AAV vector genome can be based upon any AAV genome, such as AAV-1, -2, -3, -4, -5, -6, -7, -8, -9, -10, -11, -rh74, -rh10 or AAV-2i8, for example. Such vectors can be based on the same of strain or serotype (or subgroup or variant), or be different from each other. As a non-limiting example, a recombinant AAV vector genome based upon one serotype genome can be identical to one or more of the capsid proteins that package the vector. In addition, a recombinant AAV vector genome can be based upon an AAV (e.g., AAV2) serotype genome distinct from one or more of the capsid proteins that package the vector, in which case at least one of the three capsid proteins could be a AAV1, AAV2, AAV3, AAV4, AAV5, AAV6, AAV7, AAV8, AAV9, AAV10, AAV11, Rh10, Rh74 or AAV-2i8 or variant (e.g., capsid variants such as LK03, 4-1, etc.), for example.

AAV vectors therefore include gene/protein sequences identical to gene/protein sequences characteristic for a particular serotype. As used herein, an “AAV vector related to AAV1” refers to one or more AAV proteins (e.g., VP1, VP2, and/or VP3 sequences) that has substantial sequence identity to one or more polynucleotides or polypeptide sequences that comprise AAV1. Analogously, an “AAV vector related to AAV8” refers to one or more AAV proteins (e.g., VP1, VP2, and/or VP3 sequences) that has substantial sequence identity to one or more polynucleotides or polypeptide sequences that comprise AAV8. An “AAV vector related to AAV-Rh74” refers to one or more AAV proteins (e.g., VP1, VP2, and/or VP3 sequences) that has substantial sequence identity to one or more polynucleotides or polypeptide sequences that comprise AAV-Rh74. (see, e.g., VP1, VP2, VP3). Such AAV vectors related to another serotype, e.g., AAV1, AAV2, AAV3, AAV4, AAV5, AAV6, AAV7, AAV8, AAV9, AAV10, AAV11, Rh10, Rh74 or AAV-2i8, can therefore have one or more distinct sequences from AAV1, AAV2, AAV3, AAV4, AAV5, AAV6, AAV7, AAV8, AAV9, AAV10, AAV11, Rh10, Rh74 and AAV-2i8, but can exhibit substantial sequence identity to one or more genes and/or proteins, and/or have one or more functional characteristics of AAV1, AAV2, AAV3, AAV4, AAV5, AAV6, AAV7, AAV8, AAV9, AAV10, AAV11, Rh10, Rh74 or AAV-2i8 (e.g., such as cell/tissue tropism). Exemplary non-limiting AAV-Rh74 and related AAV variants include capsid variant 4-1 in Example 6.

In various exemplary embodiments, an AAV vector related to a reference serotype has a polynucleotide, polypeptide or subsequence thereof that includes or consists of a sequence at least 70% or more (e.g., 75%, 80%, 85%, 90%, 95%, 96%, 97%, 98%, 99%, 99.5%, etc.) identical to one or more AAV1, AAV2, AAV3, AAV4, AAV5, AAV6, AAV7, AAV8, AAV9, AAV10, AAV11, Rh10, Rh74 or AAV-2i8. Accordingly, methods and uses of the invention include AAV sequences (polypeptides and nucleotides) and subsequences thereof that exhibit 100% or less than 100% sequence identity to a reference AAV serotype such as AAV1, AAV2, AAV3, AAV4, AAV5, AAV6, AAV7, AAV8, AAV9, AAV10, AAV11, Rh10, or AAV-2i8, for example, AAV-Rh74 gene or protein sequence (e.g., VP1, VP2, and/or VP3 sequences set forth Example 6), but may be distinct from and not identical to known AAV genes or proteins, such as AAV1, AAV2, AAV3, AAV4, AAV5, AAV6, AAV7, AAV8, AAV9, AAV10, AAV11, Rh10, Rh74 or AAV-2i8, genes or proteins, etc.

In one embodiment, an AAV polypeptide or subsequence thereof includes or consists of a sequence at least 70%% or more identical, e.g., 75%, 80%, 85%, 86%, 87%, 88%, 89%, 90%, 91%, 92%, 93%, 94%, 95%, 96%, 97%, 98%, 99%, 99.5%, etc., i.e. up to 100% identical to any reference AAV sequence or subsequence thereof, such as AAV1, AAV2, AAV3, AAV4, AAV5, AAV6, AAV7, AAV8, AAV9, AAV10, AAV11, Rh10, Rh74 or AAV-2i8 (e.g., VP1, VP2 and/or VP3 sequences set forth in Example 6). In particular aspects, an AAV variant comprises a capsid variant 4-1 or LK03 VP1, VP2 and/or VP3 as set forth in Example 6. Recombinant AAV vectors, variants, hybrids and chimeric sequences, can be constructed using recombinant techniques that are known to the skilled artisan, to include one or more heterologous nucleic acid sequences (transgenes) flanked with one or more functional AAV ITR sequences. Such rAAV vectors can have one or more of the wild type AAV genes deleted in whole or in part, for example, a rep and/or cap gene, but retain at least one functional flanking ITR sequence, as necessary for the rescue, replication, and packaging of the recombinant vector into an AAV vector particle. An AAV vector genome would therefore include sequences required in cis for replication and packaging (e.g., functional ITR sequences).

An “AAV ITR” or “AAV ITRs” refers to the art-recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome.

The nucleotide sequences of AAV ITRs are known. An “AAV ITR” need not have the wild-type nucleotide sequence depicted, but may be altered, e.g., by the insertion, deletion or substitution of nucleotides. Additionally, the AAV ITR may be derived from any of several AAV serotypes. Furthermore, 5′ and 3′ ITRs which flank a heterologous nucleic acid sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome.

The terms “nucleic acid” and “polynucleotide” are used interchangeably herein to refer to all forms of nucleic acid, oligonucleotides, including deoxyribonucleic acid (DNA) and ribonucleic acid (RNA). Polynucleotides include genomic DNA, cDNA and antisense DNA, and spliced or unspliced mRNA, rRNA tRNA and inhibitory DNA or RNA (RNAi, e.g., small or short hairpin (sh)RNA, microRNA (miRNA), small or short interfering (si)RNA, trans-splicing RNA, or antisense RNA). Nucleic acids include naturally occurring, synthetic, and intentionally modified or altered polynucleotides (e.g., having reduced CpG dinucleotides). Nucleic acids can be single, double, or triplex, linear or circular. In discussing nucleic acids, a sequence or structure of a particular nucleic acid may be described herein according to the convention of providing the sequence in the 5′ to 3′ direction.

rAAV vectors as set forth herein comprise an exogenous (heterologous) nucleic acid functionally linked to a promoter. A “heterologous” nucleic acid refers to a nucleic acid inserted into an AAV vector for purposes of vector mediated transfer/delivery of the nucleic acid into a cell, tissue or organ. Heterologous nucleic acids are distinct from AAV nucleic acid, i.e., are non-native with respect to AAV nucleic acid. For example, in certain embodiments, the heterologous nucleic acid encodes a protective ApoE isoform.

Although the term “heterologous” is not always used herein in reference to nucleic acid, reference to a nucleic acid even in the absence of the modifier “heterologous” is intended to include heterologous nucleic acids in spite of the omission. Once transferred/delivered into the cell, a heterologous nucleic acid, contained within the rAAV vector, can be expressed (e.g., transcribed, and translated as appropriate).

The term “transgene” is used herein to conveniently refer to a heterologous nucleic acid that is intended or has been introduced into a cell or organism. Transgenes include any nucleic acid, such as a gene that encodes a polypeptide or protein (e.g., a protective apoE isoform).

In a cell having a transgene, the transgene has been introduced/transferred by way of rAAV vector “infection” or “transduction” of the cell. The terms “infect” and “transduce” refer to introduction of a molecule such as a nucleic acid into a cell or host organism, e.g., by way of an AAV vector. An “infected” or “transduced” cell (e.g., in a mammal, such as a cell or tissue or organ cell), means a genetic change in a cell following incorporation of an nucleic acid (e.g., a transgene) into the cell. Thus, an “infected” or “transduced” cell is a cell into which, or a progeny thereof in which an exogenous molecule has been introduced, for example. The cell(s) can be propagated and the introduced nucleic acid transcribed and protein expressed. For gene therapy uses and methods, a transduced cell can be in a subject.

Cells that may be transduced include non-CNS cells, tissues or organs. CNS cells, tissues or organs include cerebrospinal fluid (CSF), brain, intracranial space, and spinal cord. Thus, reference to non-CNS cells, tissues or organs excludes cerebrospinal fluid (CSF), brain, intracranial space, and spinal cord.

Cells that may be transduced also include non-ocular cells, tissues or organs. Ocular cells, tissues and organs include eye and parts of the eye. Thus, reference to non-ocular cells, tissues or organs excludes the eye and parts of the eye.

Non-limiting examples of non-CNS and non-ocular cells include liver (e.g., hepatocytes, sinusoidal endothelial cells) and pancreas (e.g., beta islet cells). Other examples include skeletal muscle cells (e.g., fibroblasts).

The “polypeptides,” “proteins” and “peptides” encoded by the “nucleic acid sequences” include full-length native sequences, as with naturally occurring proteins, as well as functional subsequences, modified forms or sequence variants so long as the subsequence, modified form or variant retains some degree of functionality of the native full-length protein. In methods and uses of the invention, such polypeptides, proteins and peptides encoded by the nucleic acid sequences can be but are not required to be identical to the endogenous protein that is defective, or whose expression is insufficient, or deficient in the treated mammal.

A “therapeutic molecule” in one embodiment is a peptide or protein that may alleviate or reduce symptoms that result from an absence or defect in a protein in a cell or subject. Alternatively, a “therapeutic” peptide or protein encoded by a transgene is one that confers a benefit to a subject, e.g., to correct a genetic defect, to correct a gene (expression or functional) deficiency.

Non-limiting examples of heterologous nucleic acids encoding therapeutic proteins useful in accordance with the invention include those that may be used in the treatment of a disease or disorder including, but not limited to a CNS disease or disorder. Particular non-limiting examples include a protective ApoE isoform, e.g., a sequence at least 70% identical to human ApoE ε2; TPP1, e.g., a sequence at least 70% identical to human TPP1; CLN3, e.g., a sequence at least 70% identical to human CLN3; PPT1, e.g., a sequence at least 70% identical to human PPT1; CLN6, e.g., a sequence at least 70% identical to human CLN6; and CLN8 e.g., a sequence at least 70% identical to human CLN8. Further particular non-limiting examples include Galactosamine-6-sulfatase, e.g., a sequence at least 70% identical to human Galactosamine-6-sulfatase and beta-glucuronidase, e.g., a sequence at least 70% identical to human beta-glucuronidase.

Non-limiting examples of a CNS disease or disorder include Alzheimer's disease, a lysosomal storage disease, neuronal ceroid lipofuscinosis (NCL), such as infantile NCL, late infantile NCL, juvenile NCL (Batten disease), adult NCL, or Mucopolysaccharidosis (e.g., MPS IV or MPS VII).

rAAV vectors as set forth herein optionally further include additional elements, such as an expression control element (e.g., a promoter, enhancer), intron, ITR(s), poly-Adenine (also referred to as poly-adenylation) sequence. Typically, expression control elements are sequence(s) that influence expression of an operably linked nucleic acid. Control elements, including expression control elements as set forth herein such as promoters and enhancers, present within a vector are included to facilitate proper heterologous nucleic acid transcription and if appropriate translation (e.g., a promoter, enhancer, splicing signal for introns, maintenance of the correct reading frame of the gene to permit in-frame translation of mRNA and, stop codons etc.). Such elements typically act in cis, referred to as a “cis acting” element, but may also act in trans.

Expression control can be effected at the level of transcription, translation, splicing, message stability, etc. Typically, an expression control element that modulates transcription is juxtaposed near the 5′ end (i.e., “upstream”) of a transcribed nucleic acid. Expression control elements can also be located at the 3′ end (i.e., “downstream”) of the transcribed sequence or within the transcript (e.g., in an intron). Expression control elements can be located adjacent to or at a distance away from the transcribed sequence (e.g., 1-10, 10-25, 25-50, 50-100, 100 to 500, or more nucleotides from the polynucleotide), even at considerable distances. Nevertheless, owing to the length limitations of certain vectors, such as AAV vectors, such expression control elements will typically be within 1 to 1000 nucleotides from the transcribed nucleic acid.

Functionally, expression of operably linked heterologous nucleic acid is at least in part controllable by the element (e.g., promoter) such that the element modulates transcription of the polynucleotide and, as appropriate, translation of the transcript. A specific example of an expression control element is a promoter, which is usually located 5′ of the transcribed sequence. Another example of an expression control element is an enhancer, which can be located 5′, 3′ of the transcribed sequence, or within the transcribed sequence.

A “promoter” as used herein can refer to a nucleic acid (e.g., DNA) sequence that is located adjacent to a polynucleotide sequence that encodes a recombinant product. A promoter is typically operatively linked to an adjacent sequence, e.g., heterologous nucleic acid. A promoter typically increases an amount expressed from a heterologous polynucleotide as compared to an amount expressed when no promoter exists.

An “enhancer” as used herein can refer to a sequence that is located adjacent to the heterologous polynucleotide. Enhancer elements are typically located upstream of a promoter element but also function and can be located downstream of or within a DNA sequence (e.g., a heterologous nucleic acid). Hence, an enhancer element can be located 100 base pairs, 200 base pairs, or 300 or more base pairs upstream or downstream of a heterologous nucleic acid. Enhancer elements typically increase expressed of a heterologous nucleic acid above increased expression afforded by a promoter element.

Expression control elements (e.g., promoters) include those active in a particular tissue or cell type, referred to herein as a “tissue-specific expression control elements/promoters.” Tissue-specific expression control elements are typically active in specific cell or tissue (e.g., liver, pancreas, muscle, etc.). Expression control elements are typically active in these cells, tissues or organs because they are recognized by transcriptional activator proteins, or other regulators of transcription, that are unique to a specific cell, tissue or organ type.

The promoter can be any desired promoter, selected by known considerations, such as the level of expression of a nucleic acid functionally linked to the promoter and the cell type in which the vector is to be used. Promoters can be an exogenous or an endogenous promoter.

Examples of promoters active in skeletal muscle include promoters from genes encoding skeletal α-actin, myosin light chain 2A, dystrophin, muscle creatine kinase, as well as synthetic muscle promoters with activities higher than naturally-occurring promoters (see, e.g., Li, et al., Nat. Biotech. 17:241-245 (1999)). Examples of promoters that are tissue-specific for liver are the human alpha 1-antitrypsin (hAAT) promoter; albumin, Miyatake, et al. J. Virol., 71:5124-32 (1997); hepatitis B virus core promoter, Sandig, et al., Gene Ther. 3:1002-9 (1996); alpha-fetoprotein (AFP), Arbuthnot, et al., Hum. Gene. Ther., 7:1503-14 (1996)], bone (osteocalcin, Stein, et al., Mol. Biol. Rep., 24:185-96 (1997); bone sialoprotein, Chen, et al., J. Bone Miner. Res. 11:654-64 (1996)), lymphocytes (CD2, Hansal, et al., J. Immunol., 161:1063-8 (1998); immunoglobulin heavy chain; T cell receptor a chain), and TTR promoter. An example of an enhancer active in liver is apolipoprotein E (apoE) HCR-1 and HCR-2 (Allan et al., J. Biol. Chem., 272:29113-19 (1997)).

Expression control elements also include ubiquitous or promiscuous promoters/enhancers which are capable of driving expression of a polynucleotide in many different cell types. Such elements include, but are not limited to viral promoters such as the cytomegalovirus (CMV) immediate early promoter/enhancer sequences, the Rous sarcoma virus (RSV) promoter/enhancer sequences and the other viral promoters/enhancers active in a variety of mammalian cell types, or synthetic elements that are not present in nature (see, e.g., Boshart et al, Cell, 41:521-530 (1985)), the SV40 promoter, bovine papilloma virus promoter, the dihydrofolate reductase promoter, the cytoplasmic β-actin promoter and the phosphoglycerol kinase (PGK) promoter. Additional promoters include the inducible metallothionein promoter, an AAV promoter, such as an AAV p5 promoter, promoters derived from actin genes, immunoglobulin genes, adenoviral promoters, such as the adenoviral major late promoter, an inducible heat shock promoter, respiratory syncytial virus, etc.

Expression control elements also can confer expression in a manner that is regulatable, that is, a signal or stimuli increases or decreases expression of the operably linked heterologous polynucleotide. A regulatable element that increases expression of the operably linked polynucleotide in response to a signal or stimuli is also referred to as an “inducible element” (i.e., is induced by a signal). Particular examples include, but are not limited to, a hormone (e.g., steroid) inducible promoter. A regulatable element that decreases expression of the operably linked polynucleotide in response to a signal or stimuli is referred to as a “repressible element” (i.e., the signal decreases expression such that when the signal, is removed or absent, expression is increased). Typically, the amount of increase or decrease conferred by such elements is proportional to the amount of signal or stimuli present; the greater the amount of signal or stimuli, the greater the increase or decrease in expression. Particular non-limiting examples include zinc-inducible sheep metallothionine (MT) promoter; the steroid hormone-inducible mouse mammary tumor virus (MMTV) promoter; the T7 polymerase promoter system (WO 98/10088); the tetracycline-repressible system (Gossen, et al., Proc. Natl. Acad. Sci. USA, 89:5547-5551 (1992)); the tetracycline-inducible system (Gossen, et al., Science. 268:1766-1769 (1995); see also Harvey, et al., Curr. Opin. Chem. Biol. 2:512-518 (1998)); the RU486-inducible system (Wang, et al., Nat. Biotech. 15:239-243 (1997) and Wang, et al., Gene Ther. 4:432-441 (1997)]; and the rapamycin-inducible system (Magari, et al., J. Clin. Invest. 100:2865-2872 (1997); Rivera, et al., Nat. Medicine. 2:1028-1032 (1996)). Other regulatable control elements which may be useful in this context are those which are regulated by a specific physiological state, e.g., temperature, acute phase, development.

As used herein, the term “operable linkage” or “operably linked” refers to a physical or functional juxtaposition of the components so described as to permit them to function in their intended manner. In the example of an expression control element in operable linkage with a nucleic acid, the relationship is such that the control element modulates expression of the nucleic acid. More specifically, for example, two DNA sequences operably linked means that the two DNAs are arranged (cis or trans) in such a relationship that at least one of the DNA sequences is able to exert a physiological effect upon the other sequence.

Additional elements that rAAV vectors and plasmids include, for example, filler or stuffer polynucleotide sequences, for example to improve packaging and reduce the presence of contaminating nucleic acid, e.g., to reduce packaging of the plasmid backbone. AAV vectors typically accept inserts of DNA having a defined size range which is generally about 4 kb to about 5.2 kb, or slightly more. Thus, for shorter sequences, inclusion of a stuffer or filler in the insert fragment in order to adjust the length to near or at the normal size of the virus genomic sequence acceptable for AAV vector packaging into virus particle. In various embodiments, a filler/stuffer nucleic acid sequence is an untranslated (non-protein encoding) segment of nucleic acid. In particular embodiments of an AAV vector, a heterologous polynucleotide sequence has a length less than 4.7 kb and the filler or stuffer polynucleotide sequence has a length that when combined (e.g., inserted into a vector) with the heterologous polynucleotide sequence has a total length between about 3.0-5.5 kb, or between about 4.0-5.0 Kb, or between about 4.3-4.8 Kb.

AAV “empty capsids” as used herein do not contain a vector genome (hence, the term “empty”), in contrast to “genome containing capsids” which contain an AAV vector genome. Empty capsids are virus-like particles in that they react with one or more antibodies that reacts with the intact (genome containing AAV vector) virus.

Although not wishing to be bound by theory, AAV empty capsids are believed to bind to or react with antibodies against the AAV vectors, thereby functioning as a decoy to reduce inactivation of the AAV vector. Such a decoy acts to absorb antibodies directed against the AAV vector thereby increasing or improving AAV vector transgene transduction of cells (introduction of the transgene), and in turn increased cellular expression of the transcript and/or encoded protein.

Empty capsids can be generated and purified at a quality and their quantities determined. For example, empty capsid titer can be measured by spectrophotometry by optical density at 280 nm wavelength (based on Sommer et al., Mol. Ther. 2003 January; 7(1):122-8).

Empty-AAV or empty capsids are sometimes naturally found in AAV vector preparations. Such natural mixtures can be used in accordance with the invention, or if desired be manipulated to increase or decrease the amount of empty capsid and/or vector. For example, the amount of empty capsid can optionally be adjusted to an amount that would be expected to reduce the inhibitory effect of antibodies that react with an AAV vector that is intended to be used for vector-mediated gene transduction in the subject. The use of empty capsids is described in US Publication 2014/0336245.

In various embodiments, AAV empty capsids are formulated with rAAV vectors and/or administered to a subject. In particular aspects, AAV empty capsids are formulated with less than or an equal amount of vector (e.g., about 1.0 to 100-fold AAV vectors to AAV empty capsids, or about a 1:1 ratio of AAV vectors to AAV empty capsids). In other particular aspects, AAV vectors are formulated with an excess of AAV empty capsids (e.g., greater than 1 fold AAV empty capsids to AAV vectors, e.g., 1.0 to 100-fold AAV empty capsids to AAV vectors).

In some embodiments, empty capsids comprise the same VP1, VP2, and VP3 capsid proteins that are present in the rAAV vectors. In other embodiments, empty capsids comprise VP1, VP2 and VP3 proteins having a different amino acid sequence than those found in the rAAV vectors. Typically, although not necessarily, if the capsid proteins of the empty capsids and capsids of the rAAV vectors are not identical in sequence, they will be of the same serotype.

Suitable mammals include humans, non-human primates (apes, gibbons, gorillas, chimpanzees, orangutans, macaques), a domestic animal (dogs and cats), a farm animal (poultry such as chickens and ducks, horses, cows, goats, sheep, pigs), and experimental animals (mouse, rat, rabbit, guinea pig). Humans include fetal, neonatal, infant, juvenile and adult subjects. Animal disease models include, for example, mouse and other mammalian models known to those of skill in the art.

A mammal appropriate for treatment include those having or at risk of producing an insufficient amount or having a deficiency in a functional gene product (protein), or produce an aberrant, partially functional or non-functional gene product (protein), which can lead to disease. Subjects appropriate for treatment in accordance with the invention also include those having or at risk of producing an aberrant, or defective (mutant) gene product (protein) that leads to a disease such that reducing amounts, expression or function of the aberrant, or defective (mutant) gene product (protein) would lead to treatment of the disease, or reduce one or more symptoms or ameliorate the disease.

Accordingly, the mammal may have a condition that is amenable to gene replacement therapy. As used herein, “gene replacement therapy” refers to administration to the recipient of nucleic acid encoding a protein and subsequent expression of the administered nucleic acid in situ. Thus, the phrase “condition amenable to gene replacement therapy” embraces conditions such as genetic diseases (i.e., a disease condition that is attributable to one or more gene defects). According to one embodiment, the mammalian recipient has a genetic disease and the rAAV vector comprises a heterologous nucleic acid encoding a therapeutic protein for treating the disease.

The invention provides methods of delivering a nucleic acid to a non-CNS cell, tissue or organ and methods of delivering a nucleic acid to a non-ocular cell, tissue or organ comprising administering to the cell, tissue or organ a rAAV particle containing a vector comprising the nucleic acid inserted between a pair of AAV inverted terminal repeats, thereby delivering the nucleic acid to the cell, tissue or organ. The rAAV particle can be allowed to remain in contact with the cells for any desired length of time, and typically the particle is administered and allowed to remain indefinitely. Administration to the cell can be accomplished by any means, including local or regional, or systemic, provided that it is not administered into the CNS and/or not to ocular cells, tissue or organs.

The rAAV vector may comprise a heterologous nucleic acid that encodes a protective ApoE isoform protein. The rAAV vector infects non-CNS and or non-ocular cells and the protective ApoE isoform protein is expressed and secreted. The expressed and secreted protective ApoE isoform protein enters into the circulation and in turn enters the CNS.

rAAV expression vectors can be constructed using known techniques to at least provide as operatively linked components in the direction of transcription, control elements including a transcriptional initiation region, the DNA of interest and a transcriptional termination region. The control elements are selected to be functional in a mammalian cell. The resulting construct which contains the operatively linked components is flanked (5′ and 3′) with functional AAV ITR sequences.

To produce rAAV virions, an AAV expression vector is introduced into a suitable host cell using known techniques, such as by transfection. A number of transfection techniques are generally known in the art. See, e.g., Sambrook et al. (1989) Molecular Cloning, a laboratory manual, Cold Spring Harbor Laboratories, New York. Particularly suitable transfection methods include calcium phosphate co-precipitation, direct micro-injection into cultured cells, electroporation, liposome mediated gene transfer, lipid-mediated transduction, and nucleic acid delivery using high-velocity microprojectiles.

By “AAV rep coding region” is the region of the AAV genome which encodes the replication proteins Rep 78, Rep 68, Rep 52 and Rep 40. These Rep expression products have been shown to possess many functions, including recognition, binding and nicking of the AAV origin of DNA replication, DNA helicase activity and modulation of transcription from AAV (or other heterologous) promoters. The Rep expression products are collectively required for replicating the AAV genome. Suitable homologues of the AAV rep coding region include the human herpesvirus 6 (HHV-6) rep gene, which is also known to mediate AAV2 DNA replication.

AAV helper functions can introduced into a host cell by transfecting the host cell with an AAV helper construct either prior to, or concurrently with, the transfection of the AAV expression vector. AAV helper constructs are thus used to provide at least transient expression of AAV rep and/or cap genes to complement missing AAV functions that are necessary for productive AAV infection. AAV helper constructs lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. A number of other vectors have been described which encode Rep and/or Cap expression products.

Invention rAAV vectors, compositions, agents, drugs, biologics (proteins) can be incorporated into pharmaceutical compositions, e.g., a pharmaceutically acceptable carrier or excipient. Such pharmaceutical compositions are useful for, among other things, administration and delivery to a subject in vivo.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All applications, publications, patents and other references, GenBank citations and ATCC citations cited herein are incorporated by reference in their entirety. In case of conflict, the specification, including definitions, will control.

All of the features disclosed herein may be combined in any combination. Each feature disclosed in the specification may be replaced by an alternative feature serving a same, equivalent, or similar purpose. Thus, unless expressly stated otherwise, disclosed features (e.g., modified nucleic acid, vector, plasmid, a recombinant AAV (rAAV) vector, vector genome, or rAAV virus particle) are an example of a genus of equivalent or similar features.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Thus, such a pharmaceutical composition may be used, for example in administering a rAAV vector or rAAV particle to a subject.

Such compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions include carriers, diluents, or excipients suitable for administration by various routes. Compositions suitable for parenteral administration comprise aqueous and non-aqueous solutions, suspensions or emulsions of the active compound, which preparations are typically sterile and can be isotonic with the blood of the intended recipient. Non-limiting illustrative examples include water, saline, dextrose, fructose, ethanol, animal, vegetable or synthetic oils.

Pharmaceutical compositions and delivery systems appropriate for the compositions, methods and uses of the invention are known in the art (see, e.g., Remington: The Science and Practice of Pharmacy (2003) 20^{th} ed., Mack Publishing Co., Easton, Pa.; Remington's Pharmaceutical Sciences (1990) 18^{th} ed., Mack Publishing Co., Easton, Pa.; The Merck Index (1996) 12^{th} ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11^{th} ed., Lippincott Williams & Wilkins, Baltimore, Md.; and Poznansky et al., Drug Delivery Systems (1980), R. L. Juliano, ed., Oxford, N.Y., pp. 253-315).

A “unit dose” or a “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. rAAV vectors, rAAV particles, and pharmaceutical compositions thereof can be packaged in single or multiple unit dosage form for ease of administration and uniformity of dosage.

A variety of detection methods can be used to ascertain the state of a disease or improvement. Such detection methods include immunodetection. Immunodetection methods include enzyme linked immunosorbent assay (ELISA), radioimmunoassay (RIA), immunoradiometric assay, fluoroimmunoassay, chemiluminescent assay, bioluminescent assay, and Western blot. Others are known to those of ordinary skill. Various useful immunodetection methods have been described in the scientific literature.

In general, immunobinding methods include obtaining a sample suspected of containing Aβ protein and contacting the sample with a first antibody, monoclonal or polyclonal specific for Aβ, as the case may be, under conditions effective to allow the formation of immunocomplexes.

The immunobinding methods include methods for detecting and/or quantifying the amount of Aβ protein in a sample and the detection and/or quantification of any immune complexes formed during the binding process. Here, one could obtain a sample suspected of containing Aβ protein, and contact the sample with an antibody and then detect and quantify the amount of immune complexes formed under the specific conditions.

Contacting a biological sample with the antibody under conditions and for a period of time sufficient to allow the formation of immune complexes (primary immune complexes) is generally a matter of simply adding the antibody composition to the sample and incubating the mixture for a period of time so the antibodies form immune complexes with, i.e., to bind to, any antigens present. After this time, the sample-antibody composition, such as blood, plasma or serum samples, or a tissue section, ELISA plate, dot blot or western blot, will generally be processed (e.g., washed) to remove any non-specifically bound antibody species, allowing only those molecules specifically bound within the primary immune complexes to be detected.

In general, the detection of immunocomplex formation is known in the art and may be achieved through the application of numerous approaches. These methods are generally based upon the detection of a label or marker, such as any of those radioactive, fluorescent, biological and enzymatic tags. U.S. patents concerning the use of such labels include U.S. Pat. Nos. 3,817,837; 3,850,752; 3,939,350; 3,996,345; 4,277,437; 4,275,149 and 4,366,241. Of course, one may employ the use of a secondary binding ligand such as a second antibody and/or a biotin/avidin ligand binding arrangement, as is known in the art.

As noted above, a protein detection molecule (i.e., binding ligand, such as an antibody or antibody fragment) may itself be linked to a detectable label, wherein one would then simply detect this label, thereby allowing the amount of the primary immune complexes in the composition to be determined and/or quantified. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include detection of primary immune complexes by a two-step approach. A second binding ligand, such as an antibody, that has binding affinity for the first binding ligand is used to form secondary immune complexes, as described above. After washing, the secondary immune complexes are contacted with a third binding ligand or antibody that has binding affinity for the second antibody, again under conditions and for a period of time sufficient to allow formation of immune complexes (tertiary immune complexes). The third ligand or antibody may be linked to a detectable label, allowing detection of the tertiary immune complexes formed. This system may provide for signal amplification if desired.

As detailed above, immunoassays are binding assays. Certain immunoassays are the various types of enzyme linked immunosorbent assays (ELISAs) and/or radioimmunoassays (RIA) known in the art. Immunohistochemical detection using tissue sections is also useful. It will be readily appreciated that detection is not limited to such techniques, and/or western blotting, dot blotting, FACS analyses, and/or the like may also be used.

In an exemplary ELISA, antibodies are immobilized onto a selected surface exhibiting protein affinity, such as a well in a microtiter plate. Then, a test composition suspected of containing AP protein, such as a clinical sample (e.g., a biological sample obtained from the subject), is added to the wells. After binding and/or washing to remove non-specifically bound immune complexes, antibody bound antigen may be detected. Detection is generally achieved by the addition of another (secondary) antibody that is linked to a detectable label. This type of ELISA is a simple “sandwich ELISA.” Detection may also be achieved by the addition of a second antibody, followed by the addition of a third antibody that has binding affinity for the second antibody, with the third antibody being linked to a detectable label.

In another exemplary ELISA, the samples suspected of containing the antigen are immobilized onto the well surface and/or then contacted with binding agents. After binding and/or washing to remove non-specifically bound immune complexes, the bound anti-binding agents are detected. Where the initial binding agents are linked to a detectable label, the immune complexes may be detected directly. Again, the immune complexes may be detected using a second antibody that has binding affinity for the first binding agents, with the second antibody being linked to a detectable label.

Another ELISA, in which the antigens are immobilized, involves the use of antibody competition for detection. In this ELISA, labeled antibodies against an antigen are added to the wells, allowed to bind, and/or detected by means of their label. The amount of an antigen in an unknown sample is then determined by mixing the sample with the labeled antibodies against the antigen during incubation with coated wells. The presence of antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting and/or quantitating the bound immune complexes.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then employ a labeled secondary binding ligand or antibody, and a secondary binding ligand or antibody in conjunction with a labeled tertiary antibody or a third binding ligand, and so forth.

“Under conditions to allow immune complex (antigen/antibody) formation” means that the conditions that permit or facilitate binding. Such conditions can include diluting sample, such as AP protein, tau oligomers, etc., and/or antibody composition with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow binding. Exemplary non-limiting incubation steps typically are from about 1 to 2 to 4 hours or so, at temperatures preferably on the order of 25° C. to 27° C., or may be overnight at about 4° C. or so.

Following all incubation steps in an ELISA, the contacted surface is washed to remove non-complexed material. An example of a washing procedure includes washing with a solution such as PBS/Tween, or borate buffer. Following the formation of specific immune complexes between the test sample and the originally bound material, and subsequent washing, even minute amounts of immune complexes may be determined.

To provide a detecting means, the second or third antibody can have an associated label to provide detection. This may be an enzyme that will generate color development upon incubating with an appropriate chromogenic substrate. Thus, for example, one can contact or incubate the first and second immune complex with a unease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

As used herein, the singular forms “a”, “and,” and “the” include plural referents unless the context clearly indicates otherwise. Thus, for example, reference to “a nucleic acid” includes a plurality of such nucleic acids, reference to “a vector” includes a plurality of such vectors, and reference to “a virus” or “particle” includes a plurality of such virions/particles.

As used herein, all numerical values or numerical ranges include integers within such ranges and fractions of the values or the integers within ranges unless the context clearly indicates otherwise. Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions. Accordingly, the following examples are intended to illustrate but not limit the scope of the invention claimed.

### EXAMPLES

### Example 1 Below is Described in WO 2015/077473: Changes in the Progression of Amyloid Deposition

This Example studied changes in the progression of amyloid deposition in app/ps mice after overexpression of different ApoE isoforms through intraventricular injection of an adeno-associated virus serotype 4 (AAV4).

The epsilon 4 allele of ApoE (ApoE ε4) is the first genetic risk factor for Alzheimer disease (AD), whereas inheritance of the rare epsilon 2 allele of ApoE (ApoE ε2) reduces this risk by about half. However, despite the discovery of these strong genetic clues almost 17 years ago, the mechanisms whereby ApoE confer risk remains uncertain.

In order to decipher how the different ApoE isoforms (ApoE ε2, ε3 and ε4) impact the formation and stability of fibrillar amyloid plaques, AAV4 vectors coding for each ApoE isoform were injected into the ventricle of 7 month-old APP/PS mice. Using in vivo multiphoton imaging, populations of amyloid deposits were tracked at baseline and after exposure to ApoE over a two-month interval, thus allowing a dynamic view of amyloidosis progression in a living animal.

The kinetic of amyloid plaque deposition was observed to be variable according to each isoform, so that ApoE ε4 injected mice had a 38% increase of senile plaques whereas mice treated with ApoE ε2 presented a 15% decrease of the number of amyloid deposits compared to ApoE ε3 after 2 months. The post-mortem analysis confirmed these results and revealed the presence of human ApoE proteins decorating plaques in the cortex, reflecting a large diffusion of the protein throughout the parenchyma and its focal accumulation where Aβ peptides are deposited. It is important to note that this increased content of ApoE ε4 protein was also associated with a more severe synapse loss around the amyloid deposits.

Overall, the present data demonstrated that over-production of different ApoE isoforms was able to influence the progression of the disease and could modulate the extent of synapse loss, one of the parameters that correlates best with cognitive impairment in AD patients

1. Intraventricular Injection of AAV4-ApoE LED to a Stable Expression of huApoE and a Sustained Detection of Recombinant Human ApoE (huApoE) Protein in the Brain.

Briefly, GFP and huApoE were immunodetected in APP/PS mice injected with AAV4 vectors. GFP signal could be observed into the entire ventricle area (upper panel) and in the cells lining the ventricle, as well as human APOE.

In order to evaluate the present approach, AAV4-Venus (control), -ApoE2, -ApoE3 and -ApoE4 were injected in the ventricle of wild-type mice. Two months after injection, human ApoE proteins could be detected in the cortical parenchyma around amyloid deposits (note the 3H1 antibody; only nonspecific background was observed in AAV4-GFP injected mice). Thus, a significant level of human ApoE was detected by ELISA in the brain, and immunohistological stainings for Venus and ApoE confirmed the expression of the different transgenes by the cells lining the ventricle.

qRT-PCR experiments were performed in order to evaluate the mRNA levels of the transgene. A standard curve lowed us to determine the concentrations of huApoE mRNA according to the level of endogenous GAPDH. Samples from mice that were exposed for 2 or 5 months were included. An ELISA assay designed to specifically detect human APOE was performed on brain homogenates (FIG. 1A as shown in WO 2015/077473). Low levels of recombinant proteins could be detected in AAV4-APOE injected mice compared with AAV4-GFP treated animals, as quantified by ELISA specific for human APOE (FIG. 1B as shown in WO 2015/077473) and confirmed by Western blot.

2. Overexpression of Each Isoform of APOE Differentially Affects the Progression of the Amyloidosis.

In vivo two-photon imaging was used to follow amyloid deposition over time in a living animal. Briefly, APP/PS mice (7 month old) were stereotactically injected with AAV4 vectors coding for ApoE2, ApoE3, ApoE4 and Venus. After 1 week, a cranial window was implanted and amyloid deposits were imaged over time after craniotomy. After 2 months, the animals were sacrificed and post-mortem analyses were performed.

2-photon images of APP/PS mice injected with AAV4-ApoE2, AAV4-ApoE3 or AAV4-ApoE4 were prepared. Amyloid plaques could be detected after intraperitoneal injection of Methoxy-X04 (5 mg/kg) and Texas Red dextran (70,000 Da molecular weight; 12.5 mg/ml in sterile PBS) was injected into a lateral tail vein to provide a fluorescent angiogram. Images were taken one week (=T0), one month and two months after injection. The same fields were captured over time to follow the progression of the lesions. Few new amyloid deposits appeared whereas few of them could not be detected anymore over a two-month period of time.

A complete analysis of in vivo images shows that the number of amyloid deposits increases significantly more rapidly in AAV4-ApoE4 injected APP/PS mice compared with both AAV4-ApoE3 and AAV4-Venus treated animals. By contrast, a small but significant decrease density of plaques is measured when AAV4-ApoE2 was used (FIG. 2 as shown in WO 2015/077473). A trend toward bigger plaques is observed in APP/PS mice injected with AAV4-ApoE4 (p<0.06), but overall the size of the plaques remains constant. The summarized data of in vivo imaging shows that overexpression of each APOE isoform differentially affect the progression of amyloid deposition in vivo. Injection of AAV4-ApoE2 leads to a slight decrease of amyloid density over times, whereas injection of AAV4-ApoE4 aggravates the amyloidosis.

3. The Size of Amyloid Plaques Vary According to Each ApoE Isoform.

In vivo two-photon imaging allowed the following of the changes of the size of each amyloid deposit over a period of 2 months. The size of plaques may remain stable, increase or decrease over time. The distributions of the size ratios between T1/TO and T2/T1 show that there is a shift towards bigger amyloid plaques in mice injected with an AAV4-ApoE4 compared with the other groups (FIG. 3 as shown in WO 2015/077473).

4. Post-Mortem Evaluation of Amyloid Load Confirms the Effects of ApoE2 and ApoE4 on Amyloid Deposition.

Two months after AAV4 injection, the post-mortem stereological evaluation revealed that AAV4-ApoE4 injected animals have a higher density of amyloid plaques in the cortex, whereas no difference could be detected between the other groups (FIG. 4A as shown in WO 2015/077473). This increased number of amyloid deposits is observed when plaques were labeled with ThioS or Bam10. However, no change in the ratio between Bam10 and ThioS was detected. Five months after injection, the effects of each ApoE isoform are more pronounced compared with two months (FIG. 4B as shown in WO 2015/077473). A significant increased density of deposits was observed when mice were injected with AAV4-ApoE4 whereas an inverse effect was detected with ApoE2. Again, no change in the ratio between Bam10 and ThioS awes detected.

5. Each ApoE Isoform Differentially Affects Synaptic Density Around Amyloid Deposits.

Array tomography was used to precisely determine the density of pre- and post-synaptic elements around amyloid deposits. This new imaging method offers capabilities for high-resolution imaging of tissue molecular architecture. Array tomography is based on ultrathin sectioning of the specimen (70 nm), immunostaining and 3D reconstruction. Representative images of array tomography samples stained for amyloid plaque and the post-synaptic maker PSD95. Array tomography images show that a decreased number of the post-synaptic marker PSD95 is observed around the amyloid deposits, but this effect is abolished far from plaques. The quantification of pre- (synapsin-1) and post-synaptic markers in the vicinity or far from plaques was determined in each group of mice injected with an AAV4 (FIG. 5A-D as shown in WO 2015/077473). The extensive quantification of pre- and post-synaptic elements confirmed that a decreased density of synapsin 1 and PSD95 was associated with amyloid plaques, this effect being dramatically amplified when ApoE4 was overexpressed in the brain of APP/PS1 mice (FIG. 5C, FIG. 5D as shown in WO 2015/077473). Overexpression of ApoE4 is associated with an increased spine loss compared with the other groups in the vicinity of amyloid deposits. On the contrary, the density of synapsin puncta is higher in ApoE2 treated animals around plaques.

Conclusion

Intraventricular injections of AAV virus serotype 4 led to a sustained and chronic over-production of soluble recombinant proteins throughout the cerebral parenchyma. Overexpression of ApoE2, ApoE3 and ApoE4 differentially affected the course of the pathology in APP/PS mice, so that the progression of amyloid load was significantly increased when ApoE4 is injected compared with ApoE3. Conversely, ApoE2 was associated with protective effects and few amyloid deposits are not detectable anymore two months after injection. The post-mortem immunohistological analysis confirmed the adverse effect of ApoE4. Sustained over-production of ApoE4 exacerbated the synapse loss observed around amyloid deposits compared with ApoE3, whereas ApoE2 had a mild effect. The present study demonstrated a direct connection between ApoE isoforms, amyloidosis progression and synapse loss in vivo.

### Example 2 Below is Described in WO 2015/077473 and Shows Successful Treatment of CNS Disorders Via CSF Delivery: Treating CNS Disorders Via Cerebral Spinal Fluid (CSF) in Large Mammals

In order to achieve gene therapy for brain disorders, such as Alzheimer's disease, it needed to be determined whether long-term, steady-state levels of therapeutic enzymes could be achieved in a mammal. It was discovered that ependymal cells (cells that lie the ventricles in the brain) can be transduced and secrete a targeted enzyme into the cerebral spinal fluid (CSF). It was determined that adeno-associated virus (AAV4) can transduce the ependyma in a mouse model with high efficiency. (Davidson et al, PNAS, 28:3428-3432, 2000.) In mice there was a normalization of stored substrate levels in disease brain after AAV4 treatment.

It was investigated whether global delivery of a vector could be effectively performed in order to achieve steady-state levels of enzyme in the CSF. First, a vector needed to be found that could transduce ependymal cells (cells that line the ventricles) in the brain of larger mammals. Studies were performed in a dog model of LINCL and a non-human primate model of LINCL. The LINCL dogs are normal at birth, but develop neurological signs around 7 months, testable cognitive deficits at ˜5-6 months, seizures at 10-11 months, and progressive visual loss.

An adeno-associated virus (AAV) was selected as the vector because of its small size (20 nm), most of its genetic material can be removed (“gutted”) so that no viral genes are present, and so that it is replication incompetent. It was previously tested whether adeno-associated virus type 4 (AAV4) vectors could mediate global functional and pathological improvements in a murine model of mucopolysaccharidosis type VII (MPS VII) caused by beta-glucuronidase deficiency (Liu et al., J. Neuroscience, 25(41):9321-9327, 2005). Recombinant AAV4 vectors encoding beta-glucuronidase were injected unilaterally into the lateral ventricle of MPS VII mice with established disease. Transduced ependyma expressed high levels of recombinant enzyme, with secreted enzyme penetrating cerebral and cerebellar structures, as well as the brainstem. Immunohistochemical studies revealed close association of recombinant enzyme and brain microvasculature, indicating that beta-glucuronidase reached brain parenchyma via the perivascular spaces lining blood vessels. Aversive associative learning was tested by context fear conditioning. Compared with age-matched heterozygous controls, affected mice showed impaired conditioned fear response and context discrimination. This behavioral deficit was reversed 6 weeks after gene transfer in AAV4 beta-glucuronidase-treated MPS VII mice. The data show that ependymal cells can serve as a source of enzyme secretion into the surrounding brain parenchyma and CSF.

Surprisingly, however, when these studies were extended to large mammals (i.e., dogs and non-human primates), the AAV4 vectors were not effective in targeting the ependyma in these animals. Instead, an AAV2 vector needed to be used. Briefly, rAAV2 was generated encoding TPP1 (AAV2-CLN2), and injected intraventricularly to transduce ependyma (Liu et al., J. Neuroscience, 25(41):9321-9327, 2005). TPP1 is the enzyme deficient in LINCL. The data indicated that ependymal transduction in NHP brain resulted in a significant increase of enzyme in CSF. The results indicated elevated levels of TPP1 activity in various brain regions, where the vertical axis show % control of activity (FIG. 7 as shown in WO 2015/077473).

In the first dog that was treated, the delivery of vector was suboptimal, but still exhibited CLN2 activity in the brain. Subsequent dogs underwent ICV delivery with stereotaxy. It was found that the cognitive abilities of the treated dogs were significantly improved over a non-treated dog, as measured by T-maze performance (FIG. 8 as shown in WO 2015/077473). Further, the effects of ICV delivery of AAV2-CLN2 in the dog model of LINCL were very pronounced. In the untreated (−/−) animal, large ventricles are present, whereas the brains of the untreated control and the treated animals did not exhibit ventricles. Following delivery of AAV.TPP1 to ventricles of LINCL dogs, detectable enzyme activity was noted in various brain regions, including the cerebellum and upper spinal cord. In two living additional affected dogs, brain atrophy was significantly attenuated, longevity was increased and cognitive function was improved. Finally, in NHP, we show that this method can achieve TPP1 activity levels 2-5 fold above wildtype.

Several AAV vectors were generated and tested to determine the optimal combination of ITR and capsid. Five different combinations were produced, once it was determined that the AAV2 ITR was most effective: AAV2/1 (i.e., AAV2 ITR and AAV1 capsid), AAV2/2, AAV2/4, AAV2/5, and AAV2/8. It was discovered that AAV2/2 worked much better in the large mammals (dogs and NHP), followed by AAV2/8, AAV2/5, AAV2/1 and AAV2/4. This was quite surprising because the order of effectiveness of the viral vectors is the opposite of what was observed in mice.

Thus, the present work has shown that ventricular lining cells can be a source of recombinant enzyme in CSF for distribution throughout the brain, and that AAV2/2 is an effective vehicle for administering therapeutic agents, such as the gene encoding CLN2 (TPP1) in dogs and nonhuman primates.

### Example 3 Below is Described in WO 2015/077473 and Shows Effect of Different ApoE Isoforms Delivered by Way of an AAV Vector to the CNS: Human APOE Isoforms Delivered Via Gene Transfer Differentially Modulate Alzheimer's Disease by Affecting Amyloid Deposition, Clearance, and Neurotoxicity

Alzheimer's disease (AD) is the most frequent age-related neurodegenerative disorder and has become a major public health concern. Among the susceptibility genes associated with the late onset sporadic form of AD, the apolipoprotein E ε4 (APOE-gene; ApoE-protein) allele is by far the most significant genetic risk factor. The presence of one APOE ε4 copy substantially increases the risk to develop the disease by a factor of 3 compared with the most common APOE ε3 allele, whereas two copies lead to a 12-fold increase. Intriguingly, APOE ε2 has an opposite impact and is a protective factor, so that inheritance of this specific allele decreases the age-adjusted risk of AD by about a half compared to APOE3/3. The average age of onset of dementia also corresponds to these risk profiles, with APOE4/4 carriers having an onset in their mid-60's and APOE2/3 carriers in their early 90's, a shift of almost 3 decades, whereas APOE3/3 individuals have an age of onset in between—in the mid 1970's.

The mechanism whereby ApoE impacts AD is controversial. The accumulation of Aβ containing senile plaques in the hippocampus and cortex of patients is believed to play a central role in AD, because all the known genes responsible for the rare autosomal dominant forms of the disease participate in the production of Aβ peptides. Interestingly, APOE genotype was shown to strongly affect the extent of amyloid deposition in patients with AD as well as the amount of neurotoxic soluble oligomeric Aβ detected in autopsy samples. ApoE isoforms have been suggested to differentially influence cerebrovascular integrity and affect the efflux of Aβ peptides through the blood brain barrier, thus modulating the buildup of amyloid aggregates around blood vessels (cerebral amyloid angiopathy or CAA). In addition, ApoE has also been implicated directly in neurodegeneration and in neuronal plasticity. The effects of ApoE2 have been relatively understudied in these contexts.

Genetically engineered animals expressing human APOE2, -E3 and -E4 have a similar rank order of amyloid burden as humans, consistent with the hypothesis that different ApoE isoforms impact plaque initiation and/or growth. However, further studies are needed to dissect mechanisms of ApoE mediated effects on existing amyloid deposits and on extant neurodegeneration. To overcome this gap in knowledge, we used a gene transfer approach in which adeno-associated virus vectors expressing the various APOE alleles (or GFP control) are injected into the lateral ventricle to primarily transduce the ependyma, which then act as a biological factory to deliver ApoE within the cerebrospinal and interstitial fluids. We then used intravital multiphoton microscopy to track the effects of various ApoE isoforms on plaque formation, growth, and in the case of ApoE2, dissolution, as well as in vivo microdialysis approaches to monitor ApoE and Aβ biochemical variables in the ISF, and array tomography to evaluate changes in Aβ-associated neurotoxicity.

ApoE isoforms impact the levels of soluble oligomeric Aβ in the ISF, the pace of Aβ fibrillization and deposition, the stability of amyloid deposits once formed, their clearance, and the extent of peri-plaque neurotoxic effects. Indeed, AD mice treated with ApoE4 show an enhanced amount of soluble A13, a higher density of fibrillar plaques, an exacerbation of synaptic element loss and an increased number of neuritic dystrophies around each deposit, whereas a relative protective effect was observed with ApoE2. These data support the hypothesis that APOE alleles mediate their effect on AD primarily through Aβ, and highlight ApoE as a therapeutic target.

Results

Intraventricular Injection of AA V4-APOE Leads to Stable APOE Expression and to Sustained Production of Human ApoE in the Brain

Apolipoprotein E is a naturally secreted protein, produced mainly by astrocytes and microglial cells and can diffuse throughout the cerebral parenchyma. We took advantage of this property by injecting an AAV serotype 4 coding for GFP (control) or each APOE allele into the lateral cerebral ventricles of 7 month-old APP/PS1 mice. Considering the large cerebral areas affected by the characteristic lesions of AD, this strategy offered a great advantage compared with multiple intraparenchymal injections.

Two months after injection, transduced cells were detected in the choroid plexus and ependyma lining the ventricle, thus confirming the functionality of the AAV4 vectors. Using antibodies specific for each species, both human and murine ApoE proteins were also detected by ELISA (FIGS. 9A, 9B and 15A as shown in WO 2015/077473) and Western Blot. We observed that the concentration of human apolipoprotein E reached 20 μg/mg of total protein on average (FIG. 9A as shown in WO 2015/077473), representing about 10% of the endogenous murine apoE (FIG. 9B as shown in WO 2015/077473). The presence of this modest additional amount of human ApoE did not detectably alter the levels of endogenous murine apoE protein (FIG. 15A as shown in WO 2015/077473). A small but statistically significant decrease was observed between 2 and 5 months after the AAV4 injection (FIG. 15B as shown in WO 2015/077473). Nonetheless, the levels of human protein remained detectable compared with the control group, suggesting that AAV4-mediated transduction provided a platform for sustained production of the secreted recombinant protein throughout the parenchyma. Indeed, human ApoE proteins could be detected around amyloid deposits of APP/PS1 mice throughout the cortical mantle, where endogenous marine apoE protein is known to accumulate.

Next, we assessed the presence of human ApoE in the interstitial fluid (ISF), an extracellular compartment that also contains highly biologically active Aβ soluble species. Because of the relatively small amount of ApoE detected in the entire brain lysate, we injected several apoE KO mice with each AAV4-APOE vector, and tracked the presence of the human protein using highly sensitive but non-species specific antibodies. Using a microdialysis technique, we confirmed the presence of ApoE in the ISF of apoE KO injected animals.

Overall, these data confirm that a single intracerebroventricular injection of AAV4 was sufficient to lead to sustained production of a protein of interest throughout the entire brain parenchyma and within the ISF, and that the ependyma/choroid plexus can be used as a “biological pump” to deliver potentially therapeutic proteins to the brain.

Infusion of the ApoE Isoforms Differentially Affect Amyloid Peptides and Plaque Deposition

APP/PS1 mice were transduced with vectors expressing GFP or the various ApoE isoforms for 5 months before euthanasia. An analysis of the amyloid plaque load revealed that, after 5 months, a significant increase in the density of amyloid deposits was observed in the cortex of animals injected with the AAV4-APOE4 compared with those expressing APOE2. Plaque density in AAV4-GFP and AAV4-APOE3 treated mice were not different from one another at an intermediate level (FIG. 16A as shown in WO 2015/077473).

The concentrations of Aβ₄₀ and Aβ₄₂ peptides measured from the formic acid extracts mimicked the changes observed in the amyloid plaques content, so that an increased concentration of amyloid peptides was found in mice expressing the APOE4 allele (FIG. 16B as shown in WO 2015/077473), and an opposite effect was detected with APOE2 after 5 months. The content of Aβ₄₀ and Aβ₄₂ peptides in the TBS-soluble fraction was similarly affected by the injection of each AAV-APOE (FIG. 16C as shown in WO 2015/077473). In addition, the ratio between aggregated and soluble A13 peptides remained unchanged by ApoE exposure, thus suggesting that overexpression of each distinct human ApoE isoform concomitantly modulates both the fibrillar and soluble amyloid species.

Overexpression of each ApoE isoform for only 2 months leads to smaller effects than observed in the 5 month study. Nevertheless, a significant increase in amyloid plaque density within the cortical area of AAV4-APOE4 injected mice was observed compared with the other experimental groups (FIG. 16A as shown in WO 2015/077473). This was paralleled by the amount of Aβ contained in the formic acid fraction (FIG. 16C as shown in WO 2015/077473), demonstrating a predominant effect of this specific variant. TBS-soluble Aβ_{40/42} species only showed a tendency to be lower or higher when AAV4-APOE2 or AAV4-APOE4, respectively, was expressed for 2 months (data not shown).

To determine if the presence of human ApoE isoforms might reflect an early change in the degree of fibrillization of Aβ, we also measured the ratio between robust immunostaining for Aβ using B am10 (that labels all amyloid deposits) and Thio-S (that only stains the dense core) 2 months after injection. No change was detected among the 3 isoforms, suggesting that there was no differential effect on the distribution of the dense and diffuse amyloid deposits populations across the experimental groups in this time frame (FIG. 16B as shown in WO 2015/077473). These data indicate that a longer exposure to ApoE variants has stronger effects on amyloid deposition than shorter exposure.

It has been suggested that ApoE plays a role in Aβ transport across the blood-brain barrier. To test if exposure to the ApoE isoforms might modulate the efflux of Aβ peptides through the blood brain barrier, the concentration of Aβ₄₀ was measured in the plasma of each injected animal. We observed that the plasma content of human Aβ in both intracerebroventricularly injected AAV4-APOE3 and AAV4-APOE4 mice was lower compared with AAV4-APOE2 and AAV4-GFP (FIG. 10D as shown in WO 2015/077473). This suggests that both E3 and E4 variants help retain Aβ in the central nervous system compartment, consistent with the relative increased concentrations of Ap in cerebral parenchyma observed and with previous data suggesting enhanced half-life of Aβ due to ApoE.

APOE4 carriers are more susceptible to neurovascular dysfunction, and blood brain barrier breakdown was recently shown to be favored in APOE4 transgenic mice even in the absence of amyloid deposition. In order to assess if an intraventricular injection of an AAV4-APOE in APP/PS might compromise the integrity of the BBB, post-mortem staining with Prussian blue was performed. Despite the presence of few hemosiderin positive focal areas sparsely spread across the brain in all groups, no obvious differences were observed between any of the experimental groups of animals.

Expression of ApoE Isoforms Modulates the Kinetics of the Progression of Amyloidosis

ApoE4 was associated with an increased density of amyloid deposits, whereas the opposite effect was observed with ApoE2 after 5 months. This could reflect changes in the rates of amyloid β deposition, clearance, or both. To assess how the ApoE variants affect the dynamic progression of amyloidosis, we used in vivo two-photon imaging and followed the kinetics of amyloid plaque formation and clearance. Mice received an intraventricular injection with an AAV4 vectors at 7 months of age and a cranial window was implanted one week after injection in order to perform the first imaging session (TO). After 1 (T1) and 2 month(s) (T2), amyloid deposits were imaged in the same fields of view. Mice were euthanized for post-mortem analysis after the second imaging session.

The vast majority of amyloid deposits remained stable, although occasional new plaques could be detected in the small viewing volume over the two-month time period. Moreover, on rare occasion, a Methoxy-positive plaque that was imaged at the beginning of the experiment could not be detected after one or two month(s), suggesting that some plaques could be cleared. Over time, we observed an overall increase in the volumetric density of amyloid deposits, with the density at T2 on average 23% greater than of T1. The rate of amyloid progression was faster in ApoE4 treated APP/PS1 mice, whereas ApoE2 exposed animals had a significantly reduced amyloid deposit density relative to GFP (0.66), ApoE3 (0.67) and ApoE4 (0.74) after 2 months (FIGS. 11A,11B as shown in WO 2015/077473). Importantly, the ApoE2 changes reflect a decrease from baseline, showing directly, and for the first time, non-immune mediated active clearance of plaques. In contrast to data obtained from APOE transgenic animals, these results demonstrate that induction of a modest increase of the amount of ApoE can affect the ongoing amyloidogenic process even after amyloid deposition has already started.

We next assessed single amyloid plaque growth by measuring the ratio of the cross-sectional area of individual deposits between T1/TO and T2/T1. Differences were detected among groups at T1 (ratio T1/T0), but not at T2 (ratio T2/T1, FIG. 12 as shown in WO 2015/077473), suggesting that the presence of human ApoE variants mainly affects the plaque growth during the first month after exposure, but this parameter does not differ afterwards. In particular, the size of amyloid deposits grew significantly more in ApoE4 treated mice compared with both ApoE2 and ApoE3, suggesting that not only the number of plaques as well as their size was exacerbated by this allele. ApoE4 therefore affects both the seeding of Aβ peptides as well as the size of pre-existing plaques.

Synaptic Density Around Amyloid Deposits is Worsened by ApoE3 and ApoE4 Isoforms Compared to ApoE2

Synapse loss is a parameter that correlates best with cognitive impairment. We recently showed that the presence of ApoE4 is associated with higher levels of synaptic oligomeric Aβ in the brains of human AD patients and leads to significantly decreased synapse density around amyloid plaques compared to ApoE3 (R. M. Koffie et al., Apolipoprotein E4 effects in Alzheimer's disease are mediated by synaptotoxic oligomeric amyloid-beta. Brain 135, 2155 (July, 2012); T. Hashimoto et al., Apolipoprotein E, Especially Apolipoprotein E4, Increases the Oligomerization of Amyloid beta Peptide. J Neurosci 32, 15181 (Oct. 24, 2012)). In addition, recent in vitro evidence demonstrated that ApoE4 failed to protect against Aβ induced synapse loss (M. Buttini et al., Modulation of Alzheimer-like synaptic and cholinergic deficits in transgenic mice by human apolipoprotein E depends on isoform, aging, and overexpression of amyloid beta peptides but not on plaque formation. J Neurosci 22, 10539 (Dec. 15, 2002); A. Sen, D. L. Alkon, T. J. Nelson, Apolipoprotein E3 (ApoE3) but not ApoE4 protects against synaptic loss through increased expression of protein kinase C epsilon. J Biol Chem 287, 15947 (May 4, 2012)). We therefore hypothesized that a continuous and diffuse distribution of each ApoE isoform may not only differentially affect the kinetics of Aβ deposition and clearance in the brain of APP/PS mice, but also the integrity of synapses surrounding amyloid deposits.

The densities of pre- and post-synaptic elements (respectively synapsin-1 and PSD95) were determined using array tomography, a high-resolution technique based on immunofluorescence staining of ultrathin tissue sections (K. D. Micheva, S. J. Smith, Array tomography: a new tool for imaging the molecular architecture and ultrastructure of neural circuits. Neuron 55, 25 (Jul. 5, 2007); R. M. Koffie et al., Oligomeric amyloid beta associates with postsynaptic densities and correlates with excitatory synapse loss near senile plaques. Proc Natl Acad Sci USA, 106, 4012 (Mar. 10, 2009)). As amyloid oligomeric species were shown to be highly concentrated in the close vicinity of amyloid deposits, synapsin-1 and PSD95 puncta were quantified either far (>50 μm) or close (<50 μm) from plaques using previously established protocols (R. M. Koffie et al., Oligomeric amyloid beta associates with postsynaptic densities and correlates with excitatory synapse loss near senile plaques. Proc Nall Acad Sci USA 106, 4012 (Mar. 10, 2009)). We observed that the loss of pre-synaptic elements near plaques was exacerbated when either APOE3 or APOE4 was expressed, which was not the case after injection of AAV4-APOE2 or AAV4-GFP (FIG. 13A as shown in WO 2015/077473). By contrast, the density of post-synaptic puncta remained unchanged between GFP, ApoE2 and ApoE3 injected mice, whereas ApoE4 treated animals showed a significant loss of PSD95 around amyloid deposits, thus reinforcing the deleterious effect of ApoE4 on the neurotoxic effects of Aβ (FIG. 13C as shown in WO 2015/077473). When the density of synaptic elements was evaluated in areas located far from amyloid deposits (>50 μm), no difference could be detected between the groups, suggesting that there is no effect of the human ApoE variants per se on synaptic density, but an important effect of ApoE isoforms on Aβ induced neurotoxicity. The relative synaptic loss observed with ApoE3 and ApoE4 is therefore directly related to the presence of Aβ peptides surrounding each plaque (at a distance<50 μm from its edge).

As an additional neuropathological parameter, we also evaluated the number of neuritic dystrophies associated with amyloid deposits in AAV4 injected APP/PS1 mice. In addition to a decreased spine density around them, senile plaques also cause a more general alteration of the neuropil with an increase of neurite curvature and the appearance of swollen dystrophies. These pathological changes are likely attributable to soluble oligomeric Aβ species that are enriched in a region within 50 μm of the plaque surface. We observed that overexpression of ApoE4 exacerbates the formation of SMI312-positive neuritic dystrophies associated with amyloid deposits compared with GFP, ApoE2 and ApoE3 (FIG. 13C as shown in WO 2015/077473). This result confirms the observation that the ApoE4 isoform has the strongest effect and not only modulates plaque formation but also affects amyloid associated neurotoxicity.

Human ApoE Proteins Modify the Amount Oligomeric Aβ Species Contained in the Interstitial Fluid in Another Mouse Model of AD

We next addressed the question of whether the presence of different ApoE isoforms within the ISF may alter the amount of soluble amyloid species in that same extracellular compartment. We chose to inject another model of AD, the Tg2576 mice, in order to validate our previous findings in a different transgenic mouse line. Tg2576 mice overexpress the mutated form of APP containing the Swedish mutation and present a much milder phenotype than APP/PS1 mice at a given age. We injected cohorts of 16 to 18 month old animals, so that amyloid deposits were already present at the time of AAV4-APOE transduction. Three months after gene transfer, a microdialysis probe was inserted into the hippocampus and samples were collected to characterize early changes associated with each APOE variant within the ISF.

We observed that the concentration of Aβ oligomeric species measured using the specific 82E1/82E1 ELISA assay was significantly higher (by 42±7%) after injection of the AAV4-APOE4 compared with AAV4-APOE2 (FIG. 14 as shown in WO 2015/077473), suggesting that the presence of ApoE may modulate the nature of amyloid aggregates in this extracellular compartment. Moreover, when the total Aβ₄₀ and Aβ₄₂ were assessed in the ISF, the same trends were observed but did not reach significance (FIG. 17A as shown in WO 2015/077473), suggesting that the presence of different ApoE isoforms in the ISF influences the aggregation state of amyloid peptides somewhat more than the total amount.

As expected, post-mortem biochemical analyses of brains from Tg2576 mice exposed to the various ApoE isoforms showed that the concentration of Aβ₄₂ in the formic acid fraction was significantly increased in ApoE4 treated animals (FIG. 17B as shown in WO 2015/077473), confirming in a second transgenic model our observations in APP/PS1 mice.

Taken together, these biochemical measures suggest that ApoE expression in Tg2576 mice induce similar changes in amyloid biology as observed in APP/PS1 mice. Importantly, an early change is observed in the content of oAβ within the ISF, where these neurotoxic species can directly interact with the synaptic terminals.

### Example 4: Conclusions from Examples 1-3

The foregoing studies in Examples 1-3 which are described in WO 2015/077473 show treatment efficacy of an AAV vector with a transgene encoding a protective ApoE isoform when administered into the CNS of an Alzheimer's disease animal model. The foregoing studies therefore support the proposition that a protective ApoE isoform delivered to the CNS by way of an AAV vector is a treatment of Alzheimer's disease.

### Example 5: Exemplary Assays for ApoE Detection

### APOE ELISA

Specific ELISA assays were used to detected both human and endogenous murine APOE proteins. Briefly, ELISA plates were coated overnight with 1.5 ug/ml of Goat anti-APOE antibody (to detect Murine APOE) or 1.5 ug/ml WUE4 antibody (to detect Human APOE) and blocked with 1% non-fat milk diluted in PBS for 1.5 h at 37° C. Human recombinant apoE proteins were used as standards (for human-specific assay, Biovision) or in-house mouse standards from brain extract (for the murine specific assay) and samples were diluted in ELISA buffer (0.5% BSA and 0.025% Tween-20 in PBS) and incubated overnight. After washing, detection antibodies specific for human (goat-apoe Millipore; 1:10,000) or mouse (Abcam ab20874; 1:2,000) were respectively used, followed by 1.5 h incubation with an appropriate HRP-conjugated secondary. Revelation of the signal was done using the TMB substrate before stopping the solution using H₃PO₄. The colorimetric results were measured at 450 nm.

### Aβ Quantification

The concentrations of Aβ₄₀ and Aβ₄₂ were determined by BNT-77/BA-27 (for Aβ₄₀) and BNT-77/BC-05 (for Aβ₄₂) sandwich ELISA (Wako), according to the manufacturer's instructions. Aβ oligomers were quantified using the 82E1/82E1 sandwich ELISA (Immuno-Biological Laboratories), in which the same N-terminal (residues 1-16) antibodies were used for both capture and detection (W. Xia et al., A specific enzyme-linked immunosorbent assay for measuring beta-amyloid protein oligomers in human plasma and brain tissue of patients with Alzheimer disease. Arch Neurol 66, 190 (February, 2009)).

### Example 6: Exemplary AAV Capsid Sequences

| AAV-LK03 VP1 Capsid (SEQ ID NO: 1): | |
|---|---|
| MAADGYLPDWLEDNLSEGIREWWALQPGAPKPKANQQHQDNARGLVLPGYKYLGPGNGLD | |
| KGEPVNAADAAALEHDKAYDQQLKAGDNPYLKYNHADAEFQERLKEDTSFGGNLGRAVFQ | |
| AKKRLLEPLGLVEEAAKTAPGKKRPVDQSPQEPDSSSGVGKSGKQPARKRLNFGQTGDSE | |
| SVPDPQPLGEPPAAPTSLGSNTMASGGGAPMADNNEGADGVGNSSGNWHCDSQWLGDRVI | |
| TTSTRTWALPTYNNHLYKQISSQSGASNDNHYFGYSTPWGYFDFNRFHCHFSPRDWQRLI | |
| NNNWGFRPKKLSFKLFNIQVKEVTQNDGTTTIANNLTSTVQVFTDSEYQLPYVLGSAHQG | |
| CLPPFPADVFMVPQYGYLTLNNGSQAVGRSSFYCLEYFPSQMLRTGNNFQFSYTFEDVPF | |
| HSSYAHSQSLDRLMNPLIDQYLYYLNRTQGTTSGTTNQSRLLFSQAGPQSMSLQARNWLP | |
| GPCYRQQRLSKTANDNNNSNFPWTAASKYHLNGRDSLVNPGPAMASHKDDEEKFFPMHGN | |
| LIFGKEGTTASNAELDNVMITDEEEIRTTNPVATEQYGTVANNLQSSNTAPTTRTVNDQG | |
| ALPGMVWQDRDVYLQGPIWAKIPHTDGHFHPSPLMGGFGLKHPPPQIMIKNTPVPANPPT | |
| TFSPAKFASFITQYSTGQVSVEIEWELQKENSKRWNPEIQYTSNYNKSVNVDFTVDTNGV | |
| YSEPRPIGTRYLTRPL | |
| AAV 4-1 VP1 capsid amino acid sequence (SEQ ID NO: 2): | |
| 1 | MAADGYLPDWLEDNLSEGIREWWDLKPGAPKPKANQQKQDNGRGLVLPGYKYLGPFNGLD |
| 61 | KGEPVNAADAAALEHDKAYDQQLQAGDNPYLRYNHADAEFQERLQEDTSFGGNLGRAVFQ |
| 121 | AKKRVLEPLGLVESPVKTAPGKKRPVEPSPQRSPDSSTGIGKKGQQPAKKRLNFGQTGDS |
| 181 | ESVPDPQPIGEPPAAPSGVGPNTMAAGGGAPMADNNEGADGVGSSSGNWHCDSTWLGDRV |
| 241 | ITTSTRTWALPTYNNHLYKQISNGTSGGSTNDNTYFGYSTPWGYFDFNRFHCHFSPRDWQ |
| 301 | RLINNNWGFRPKRLNFKLFNIQVKEVTQNEGTKTIANNLTSTIQVFTDSEYQLPYVLGSA |
| 361 | HQGCLPPFPADVFMIPQYGYLTLNNGSQAV GRSSFYCLEYFPSQMLRTGNNFEFSYNFED |
| 421 | VPFHSSYAHSQSLDRLMNPLIDQYLYYLSRTQSTGGTAGTQQLLFSQAGPNNMSAQAKNW |
| 481 | LPGPCYRQQRVSTTLSQNNNSNFAWTGATKYHLNGRDSLVNPGVAMATHKDDEERFFPSS |
| 541 | GVLMFGKQGAGKDNVDYSSVMLTSEEEIKTTNPVATEQYGVVADNLQQQNAAPIVGAVNS |
| 601 | QGALPGMVWQNRDVYLQGPIWAKIPHTDGNFHPSPLMGGFGLKHPPPQILIKNTPVPADP |
| 661 | PTTFNQAKLASFITQYSTGQVSVEIEWELQKENSKRWNPEIQYTSNYYKSTNVDFAVNTE |
| 721 | GTYSEPRPIGTRYLTRNL |

AAV 4-1 VP2 capsid amino acid sequence (SEQ ID NO: 3):
TAPGKKRPVEPSPQRSPDSSTGIGKKGQQPAKKRLNFGQTGDSESVPDPQPIGEPPAAPSGVGPNT
MAAGGGAPMADNNEGADGVGSSSGNWHCDSTWLGDRVITTSTRTWALPTYNNHLYKQISNGTS
GGSTNDNTYFGYSTPWGYFDFNRFHCHFSPRDWQRLINNNWGFRPKRLNFKLFNIQVKEVTQNEG
TKTIANNLTSTIQVFTDSEYQLPYVLGSAHQGCLPPFPADVFMIPQYGYLTLNNGSQAVGRSSFYCL
EYFPSQMLRTGNNFEFSYNFEDVPFHSSYAHSQSLDRLMNPLIDQYLYYLSRTQSTGGTAGTQQLLF
SQAGPNNMSAQAKNWLPGPCYRQQRVSTTLSQNNNSNFAWTGATKYHLNGRDSLVNPGVAMAT
HKDDEERFFPSSGVLMFGKQGAGKDNVDYSSVMLTSEEEIKTTNPVATEQYGVVADNLQQQNAAP
IVGAVNSQGALPGMVWQNRDVYLQGPIWAKIPHTDGNFHPSPLMGGFGLKHPPPQILIKNTPVPADPP
TTFNQAKLASFITQYSTGQVSVEIEWELQKENSKRWNPEIQYTSNYYKSTNVDFAVNTEGTYSEPRPI
GTRYLTRNL
AAV 4-1 VP3 capsid amino acid sequence (SEQ ID NO: 4):
MAAGGGAPMADNNEGADGVGSSSGNWHCDSTWLGDRVITTSTRTWALPTYNNHLYKQISNGTS
GGSTNDNTYFGYSTPWGYFDFNRFHCHFSPRDWQRLINNNWGFRPKRLNFKLFNIQVKEVTQNEG
TKTIANNLTSTIQVFTDSEYQLPYVLGSAHQGCLPPFPADVFMIPQYGYLTLNNGSQAVGRSSFYCL
EYFPSQMLRTGNNFEFSYNFEDVPFHSSYAHSQSLDRLMNPLIDQYLYYLSRTQSTGGTAGTQQLLF
SQAGPNNMSAQAKNWLPGPCYRQQRVSTTLSQNNNSNFAWTGATKYHLNGRDSLVNPGVAMAT
HKDDEERFFPSSGVLMFGKQGAGKDNVDYSSVMLTSEEEIKTTNPVATEQYGVVADNLQQQNAAP
IVGAVNSQGALPGMVWQNRDVYLQGPIWAKIPHTDGNFHPSPLMGGFGLKHPPPQILIKNTPVPADPP
TTFNQAKLASFITQYSTGQVSVEIEWELQKENSKRWNPEIQYTSNYYKSTNVDFAVNTEGTYSEPRPI
GTRYLTRNL

What is claimed is:

1. A method of delivering a therapeutic protein to the central nervous system of a mammal, comprising administering to non-central nervous system (CNS) cells, organ or tissue of the mammal a rAAV particle comprising an AAV capsid protein and a vector comprising a nucleic acid encoding the therapeutic protein inserted between a pair of AAV inverted terminal repeats in a manner effective to infect the non-CNS cells, organ or tissue in the mammal such that the non-CNS cells, organ or tissue express and secrete the therapeutic protein into the circulation of the mammal which therapeutic protein is delivered to the central nervous system of the mammal.

2. A method of treating central nervous system (CNS) disease in a mammal comprising administering to non-central nervous system (CNS) cells, organ or tissue of the mammal a rAAV particle comprising an AAV capsid protein and a vector comprising a nucleic acid encoding the therapeutic protein inserted between a pair of AAV inverted terminal repeats in a manner effective to infect the non-CNS cells, organ or tissue in the mammal such that the non-CNS cells, organ or tissue express and secrete the therapeutic protein into the circulation of the mammal which therapeutic protein is delivered to the central nervous system of the mammal.

3. A method of treating central nervous system (CNS) disease in a mammal comprising administering to non-central nervous system (CNS) cells, organ or tissue of the mammal a rAAV particle comprising an AAV capsid protein and a vector comprising a nucleic acid encoding a protective ApoE isoform between a pair of AAV inverted terminal repeats in a manner effective to infect liver cells in the mammal such that the liver cells express and secrete the protective ApoE isoform into the circulation of the mammal which protective ApoE isoform is delivered to the central nervous system of the mammal.

4. A method of treating an ocular disease in a mammal comprising administering to non-ocular cells, organ or tissue of the mammal a rAAV particle comprising an AAV capsid protein and a vector comprising a nucleic acid encoding the therapeutic protein inserted between a pair of AAV inverted terminal repeats in a manner effective to infect the non-ocular cells, organ or tissue in the mammal such that the non-ocular cells, organ or tissue express and secrete the therapeutic protein into the circulation of the mammal which therapeutic protein is delivered to the ocular organ or tissue of the mammal.

5. The method of any of claims 1-4, wherein the mammal has, is displaying one or more symptoms of or is at risk of having Alzheimer's disease, a lysosomal storage disease, neuronal ceroid lipofuscinosis (NCL), such as infantile NCL, late infantile NCL, juvenile NCL (Batten disease), adult NCL, or Mucopolysaccharidosis (e.g., MPS IV or MPS VII).

6. The method of any of claims 1-4, wherein the therapeutic protein or protective ApoE isoform is expressed at amounts providing the mammal with a beneficial or therapeutic effect.

7. The method of any of claims 1-4, wherein the therapeutic protein or protective ApoE isoform is expressed at amounts providing the mammal with a beneficial or therapeutic effect to a physical, physiological, CNS/brain patho-physiology, biochemical, histological, or behavioral characteristic of a CNS disease.

8. The method of any of claim 1-2 or 4, wherein the therapeutic protein is a protective ApoE isoform.

9. The method of claim 3 or 8, wherein the protective ApoE isoform is at least 70% identical to human ApoE ε2.

10. The method of claim 3 or 8, wherein the levels of circulating ApoE4 is reduced relative to total circulating ApoE levels.

11. The method of any of claim 1-2 or 4, wherein the therapeutic protein is TPP1.

12. The method of claim 11, wherein the TPP1 is at least 70% identical to human TPP1.

13. The method of any of claim 1-2 or 4, wherein the therapeutic protein is CLN3, PPT1, CLN6 or CLN8.

14. The method of claim 13, wherein the CLN3, PPT1, CLN6 or CLN8 is at least 70% identical to human CLN3, PPT1, CLN6 or CLN8.

15. The method of any of claims 1-5, wherein the therapeutic protein is Galactosamine-6-sulfatase.

16. The method of claim 15, wherein the Galactosamine-6-sulfatase is at least 70% identical to human Galactosamine-6-sulfatase.

17. The method of any of claims 1-5, wherein the therapeutic protein is beta-glucuronidase.

18. The method of claim 17, wherein the beta-glucuronidase is at least 70% identical to human beta-glucuronidase.

19. The method of any of claims 1-18, wherein the nucleic acid encoding the therapeutic protein has reduced cytosine-guanine dinucleotide (CpG) compared to a non-CpG reduced nucleic acid encoding the therapeutic protein.

20. The method of any of claims 1-19, wherein the nucleic acid encoding the therapeutic protein has reduced cytosine-guanine dinucleotide (CpG) compared to a wild-type nucleic acid encoding the therapeutic protein.

21. The method of any of claims 1-20, wherein the vector further comprises one or more of an intron, an expression control element, a filler polynucleotide sequence and/or poly A signal, or a combination thereof.

22. The method of any of claims 1-21, wherein the pair of ITRs are derived from or comprises a sequence of any of: AAV1, AAV2, AAV3, AAV4, AAV5, AAV6, AAV7, AAV8, AAV9, AAV10, AAV11, AAV12, LK01, LK02, LK03, AAV 4-1, AAV-2i8 ITRs, or a mixture thereof.

23. The method of any of claims 1-22, wherein the AAV capsid protein is derived from or comprise a sequence any of: AAV1, AAV2, AAV3, AAV4, AAV5, AAV6, AAV7, AAV8, AAV9, AAV10, AAV11, AAV12, LK01, LK02, LK03, AAV 4-1, AAV-2i8 ITRs, or a mixture thereof.

24. The method of any of claims 1-2, wherein the non-CNS cell, organ or tissue or non-ocular cell, organ or tissue comprises a mammalian endocrine cell, organ or tissue.

25. The method of any of claims 1-2, wherein the non-CNS cell, organ or tissue or non-ocular cell, organ or tissue comprises liver cells, liver or liver tissue and pancreas cells, pancreas or pancreatic tissue.

26. The method of any of claims 1-2, wherein the non-CNS cell, organ or tissue or non-ocular cell, organ or tissue comprises liver hepatocytes or pancreatic islets.

27. The method of claim 3, wherein the liver cells comprise liver hepatocytes.

28. The method of any of claims 1-27, wherein the rAAV particles are administered at a dose in a range from about 1×10⁸-1×10¹⁰, 1×10¹⁰-1×10¹¹, 1×10¹¹-1×10¹², 1×10¹²-1×10¹³, or 1×10¹³-1×10¹⁴ vector genomes per kilogram (vg/kg) of the mammal.

29. The method of any of claims 1-27, wherein the rAAV particles are administered at a dose of less than 1×10¹² vector genomes per kilogram (vg/kg) of the mammal.

30. The method of any of claims 1-27, wherein the rAAV particles are administered at a dose of about 5×10¹¹ vector genomes per kilogram (vg/kg) of the mammal.

31. The method of any of claims 1-27, wherein the rAAV particles administered are at least 1×10¹⁰ vector genomes (vg) per kilogram (vg/kg) of the weight of the mammal, or between about 1×10¹⁰ to 1×10¹¹ vg/kg of the weight of the mammal, or between about 1×10¹¹ to 1×10¹² vg/kg (e.g., about 1×10¹¹ to 2×10¹¹ vg/kg or about 2×10¹¹ to 3×10¹¹ vg/kg or about 3×10¹¹ to 4×10¹¹ vg/kg or about 4×10¹¹ to 5×10¹¹ vg/kg or about 5×10¹¹ to 6×10¹¹ vg/kg or about 6×10¹¹ to 7×10¹¹ vg/kg or about 7×10¹¹ to 8×10¹¹ vg/kg or about 8×10¹¹ to 9×10¹¹ vg/kg or about 9×10¹¹ to 1×10¹² vg/kg) of the weight of the mammal, or between about 1×10¹² to 1×10¹³ vg/kg of the weight of the mammal, to achieve a desired therapeutic effect.

32. The method of any of claims 1-31, wherein the mammal does not develop a substantial immune response against the therapeutic protein and/or the rAAV particle.

33. The method of any of claims 1-31, wherein the mammal does not develop a substantial humoral immune response against the therapeutic protein and/or the rAAV particle.

34. The method of any of claims 1-33, wherein the mammal does not develop a substantial immune response against the therapeutic protein and/or the rAAV particle for at least 1, 2, 3, 4, 5, 6, 7, 8, 9, 10, 11, 12, 13 or 14 continuous days, weeks or months.

35. The method of any of claims 1-31, wherein the mammal does not develop a detectable immune response against the therapeutic protein and/or the rAAV particle.

36. The method of any of claims 1-31, wherein the mammal does not develop a detectable humoral immune response against therapeutic protein and/or the rAAV particle.

37. The method of any of claims 1-31, wherein the mammal does not develop a detectable humoral immune response against therapeutic protein and/or the rAAV particle for at least 1, 2, 3, 4, 5, 6, 7, 8, 9, 10, 11, 12, 13 or 14 continuous days, weeks or months.

38. The method of any of claims 1-31, wherein the mammal does not develop an immune response against therapeutic protein and/or the rAAV particle sufficient to block a therapeutic effect of the therapeutic protein in the mammal.

39. The method of any of claims 1-31, wherein the mammal does not produce an immune response against the therapeutic protein and/or the rAAV particle sufficient to block the therapeutic effect in the mammal for at least 1, 2, 3, 4, 5, 6, 7, 8, 9, 10, 11, 12, 13 or 14 continuous days, weeks or months.

40. The method of any of claims 1-39, further comprising administration of empty AAV capsids.

41. The method of claim 40, wherein the empty AAV capsids are formulated with the rAAV particles administered to the mammal.

42. The method of any of claim 40 or 41, wherein the AAV empty capsids are administered or formulated with less than or an equal amount of rAAV vector particles.

43. The method of any of claim 40 or 41, wherein the AAV empty capsids are administered or formulated with about 1.0 to 100-fold excess of AAV empty capsids rAAV particles.

44. The method of any of claims 1-43, wherein the administering comprises infusion or injection into the systemic circulation of the subject.

45. The method of any of claims 1-43, wherein the administering comprises intravenous or intra-arterial infusion or injection into the systemic circulation of the subject.

46. The method of any of claims 1-43, wherein the administering comprises infusion or injection into the hepatic portal vein of the subject.


SEQUENCE LISTING (4 sequences)

SEQ ID NO: 1 - protein, 736 aa, Adeno-associated virus 1
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 2 - protein, 738 aa, Adeno-associated virus 1
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 3 - protein, 601 aa, Adeno-associated virus 2
TAPGKKRPVEPSPQRSPDSSTGIGKKGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 4 - protein, 535 aa, Adeno-associated virus 3
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL